SEQUENCE LISTING (13 sequences)

SEQ ID NO: 1 - DNA, 19 bp, Artificial sequence
  synthetic
tcagaagaaaaaagagaaa

SEQ ID NO: 2 - DNA, 49 bp, Artificial sequence
  synthetic
agtagaagaaaaaagagaaatgattttcatttctcttttttcttctact

SEQ ID NO: 3 - DNA, 17 bp, Artificial sequence
  synthetic
nnnnnnnnnnnnnnnnn

SEQ ID NO: 4 - DNA, 17 bp, Artificial sequence
  synthetic
nnnnnnnnnnnnnnnnn

SEQ ID NO: 5 - DNA, 17 bp, Artificial sequence
  synthetic
nnnnnnnnnnnnnnnnn

SEQ ID NO: 6 - DNA, 17 bp, Artificial sequence
  synthetic
nnnnnnnnnnnnnnnnn

SEQ ID NO: 7 - DNA, 17 bp, Artificial sequence
  synthetic
nnnnnnnnnnnnnnnnn

SEQ ID NO: 8 - DNA, 17 bp, Artificial sequence
  synthetic
nnnnnnnnnnnnnnnnn

SEQ ID NO: 9 - DNA, 17 bp, Artificial sequence
  synthetic
nnnnnnnnnnnnnnnnn

SEQ ID NO: 10 - DNA, 17 bp, Artificial sequence
  synthetic
nnnnnnnnnnnnnnnnn

SEQ ID NO: 11 - DNA, 17 bp, Artificial sequence
  synthetic
nnnnnnnnnnnnnnnnn

SEQ ID NO: 12 - DNA, 17 bp, Artificial sequence
  synthetic
nnnnnnnnnnnnnnnnn

SEQ ID NO: 13 - DNA, 17 bp, Artificial sequence
  synthetic
nnnnnnnnnnnnnnnnn

(19) United States
(12) Patent Application Publication
(10) Pub. No.: US 2004/0009602 A1
(43) Pub. Date: Jan. 15, 2004

(54) DNA modifying molecules and methods of use thereof

(75) Inventors: Michael M. Seidman, Washington, DC (US); Nitin Puri, Austin, TX (US); Alokes Majumdar, Gaithersburg, MD (US)

Correspondence Address: KLARQUIST SPARKMAN, LLP, 121 S.W. SALMON STREET, SUITE #1600, ONE WORLD TRADE CENTER, PORTLAND, OR, 97204-2988, US

(73) Assignee: The Government of the U.S.A. as represented by the Secretary of the Dept. of Health & Human Services

(21) Appl. No.: 10/438,076

(22) Filed: May 13, 2003

Related U.S. Application Data
(60) Provisional application No. 60/378,025, filed on May 13, 2002.

(51) Int. Cl. C12N 15/63; C12N 5/00

(52) U.S. Cl. 435/446; 435/325

(57) ABSTRACT

In one aspect, the disclosure concerns improved methods for the modification of genes in cells. Particular methods accomplish recombination, including targeted homologous recombination of a target gene. In another aspect, the disclosure relates to DNA modifying molecules. In one aspect, DNA modifying molecules comprise DNA targeting agents, including modified oligonucleotides, including triplex forming oligonucleotides, peptide nucleic acids and polyamides. In one embodiment, the DNA modifying molecules comprise a mutagen, and in another embodiment the DNA modifying molecules comprise a mutagen and a DNA targeting agent. The disclosure also describes methods for modifying a nucleotide sequence in the genome of a cell using the DNA targeting agents. The disclosure also relates to cells, tissue, and organisms that have been modified by DNA targeting agents.

### CROSS REFERENCE TO RELATED APPLICATION

This application claims the benefit of U.S. Provisional Application No. 60/378,025, filed May 13, 2002, which is incorporated herein by reference.

### FIELD

The disclosure concerns DNA modifying molecules and methods for using DNA modifying molecules. In one aspect, the disclosure concerns a method for using DNA modifying molecules to effect a mutation in a nucleotide sequence. In another aspect the disclosure concerns regulating gene expression using a modified oligonucleotide.

### BACKGROUND

New materials and methods for modifying genomic DNA offer numerous potential applications. For example, reagents that recognize and bind specific genomic sequences can be used for promoter suppression, gene knockout, target validation, and for genomic modifications. Site directed genomic modifications and gene therapy applications are important aspects of the new technology.

Triple helix forming oligonucleotides (TFOs) that bind chromosomal targets in living cells are useful tools for genome manipulation, including gene knockout, conversion, or recombination. However, triplex formation by DNA third strands, particularly those in the pyrimidine motif, requires non-physiological pH and Mg⁺² concentration, and this limits their development as gene targeting reagents.

The DNA triple helix has been of interest since its discovery more than four decades ago, and the recognition that it might be exploited for manipulation of the genome (1-3). Triplexes form in a sequence specific manner on polypurine:polypyrimidine tracts, which are abundant in mammalian genomes (4-6). The third strand of nucleic acid lies in the major groove of an intact duplex and is stabilized by Hoogsteen hydrogen bonds between third strand bases and the purine bases in the duplex (7, 8). Depending on the nature of the target sequences, triplexes can be formed by third strands consisting of pyrimidines or purines, and a binding code has been described (9, 10). The possibility of triple helix forming oligonucleotides (TFOs) as gene targeting reagents for application in living cells has attracted attention in recent years (11-17).

Despite this recent interest in TFOs as gene targeting agents, TFOs disclosed previously fall short of the desired biological activity due to limitations imposed by the physiological environment. In particular, pyrimidine motif triplexes are unstable at physiological pH because of the requirement for cytosine protonation that occurs at relatively acidic pH (pKa=4.5). The protonation is necessary for hydrogen bonding, and the resultant positive charge also contributes to triplex stability (18). Triplex formation requires relatively high levels of Mg⁺² to neutralize the charge-charge repulsion between the third strand and the duplex phosphates (19). These concentrations are higher than those thought to be available intracellularly (20).

Some progress towards overcoming these limitations has been achieved via the introduction of various base, sugar, and backbone modifications in triplex forming oligonucleotides (TFOs). The use of 5-methylcytosine partially alleviates the pH restriction of TFOs in the pyrimidine motif (21, 22), which has also been addressed with an adenine analog (23). Propynyl-deoxyuridine reduces the Mg⁺² dependence (24), as does replacement of the ribose with a morpholino analog (25). The recognition that RNA third strands formed more stable pyrimidine triplexes than the corresponding DNA (26) prompted the introduction of 2′-O-methoxy (2′-OMe) sugar residues which enhance triplex stability (27, 28). The 2′,4′ bridged ribose substitution also improves triplex stability, under conditions that can be physiologically relevant (29). Intercalators have been linked to TFOs to improve binding (30), a strategy employed to demonstrate targeted gene knockout in mammalian cells (16). However, these reagents still require non-physiological concentrations of a divalent cation, such as Mg²⁺ to achieve useful triplex stability. Backbone modifications that replace the phosphate linkage altogether (31, 35), or a bridging (32) oxygen with a nitrogen improve TFO activity in vitro. In particular, replacement of a non bridging oxygen in the backbone with a charged amine reduced the likelihood of self structure formation of purine TFOs in physiological K⁺ (33;34), and increased the bioactivity of a purine motif TFO (35). A positive charge on a thymidine analog (36), or linkage of positively charged moieties to TFOs, also enhance triplex stability (37;38). However, many modified oligonucleotides, particularly backbone modified oligonucleotides, are difficult to synthesize, which limits the utility of previously known modifications.

The present disclosure overcomes limitations of prior DNA modification technology by providing biostable and bioactive DNA modifying molecules. Moreover, the present disclosure describes novel methods for enhancing the activity of DNA modifying molecules and for using such molecules therapeutically.

### SUMMARY

Disclosed herein are efficient techniques and methods to enhance mutation and/or recombination rates in cell lines, tissues and organisms. In certain phases of the cell cycle, particularly S phase, the frequency of DNA modification by a DNA modifying molecule is enhanced relative to the other phases of the cell cycle. Therefore, in one particular method, a cell is treated with a DNA modifying molecule in a specific phase of the cell cycle to modify a genomic nucleotide sequence in the cell.

One aspect of the method includes synchronizing a cell population (for example, in culture) to enhance the frequency of modification induced by a DNA modifying molecule. Cell synchronization allows a DNA-modifying molecule to be provided at the optimal stage in the cell cycle, for example, at the stage where an increased frequency of DNA modification by the DNA-modifying molecule occurs. Thus, in particular embodiments, cells are contacted with a DNA-modifying molecule at a specific point in the cell cycle, wherein the points comprise interphase, prophase, metaphase, anaphase, telophase, S phase, M phase, G0 phase, G1 phase or G2 phase. In disclosed embodiments, cells are contacted at particular sub-phase of the cell cycle, for example, in the early, mid-, or late sub-phases of the above listed points in the cell cycle. In one embodiment, when cells that are substantially synchronized in S phase are contacted with the DNA-modifying molecule an increased frequency of induced mutation is observed. The observed mutation frequency is further increased when the cells are substantially synchronized in mid- to late S phase of the cell cycle.

The DNA-modifying molecule can be any molecule that provokes a mutation in a DNA sequence. DNA-modifying molecules include molecules that bind covalently or non-covalently to DNA, and further examples include radionuclides, crosslinkers, alkylators, base modifiers, DNA breakers, free radical generators and combinations thereof. Examples of DNA modifying molecules that have increased activity following activation are disclosed. Such activatable DNA modifying molecules can be activated in a specific phase of the cell cycle, thereby increasing the frequency of DNA modification. One example, without limitation, of an activation event is UV irradiation.

In particular embodiments of DNA modifying molecules, a mutagen, such as a DNA reactive group is coupled to a DNA targeting agent. Coupling of the mutagen to the DNA targeting agent enables increased frequency of induced mutation, and sequence selective mutation. The targeting agent can be any agent that has an affinity for DNA, preferably a sequence selective affinity for DNA. In one embodiment the targeting agent comprises a triplex forming oligonucleotide. In a further embodiment, the targeting agent comprises a peptide nucleic acid, and in certain embodiments the targeting agent comprises a polyamide. Additional useful DNA targeting agents include polyamide-polypyrroles, oligonucleotides designed for marker rescue and sequence specific zinc finger proteins.

Also disclosed is a method for inducing a mutation resulting in targeted homologous recombination, which introduces a competent gene in place of a defective gene. Therefore, in particular embodiments, the DNA mutagen is provided with an oligonucleotide that is at least partly homologous to a portion of the DNA of the cells.

Another aspect of the disclosure relates to DNA modifying molecules comprising novel modified oligonucleotides and methods for using modified nucleotides to, for example, inhibit expression of a particular gene, or to provoke a mutation in a particular targeted gene. The disclosed modified oligonucleotides are particularly useful for provoking mutations in synchronized cells. In one embodiment the modified oligonucleotides are modified to have enhanced affinity for a DNA target as compared to the corresponding unmodified oligonucleotides. A second type of modified oligonucleotide disclosed includes a mutagenic group for inducing or provoking a mutation in a targeted DNA sequence. In particular embodiments, the modified oligonucleotides have both an affinity-enhancing modification and a mutagenic modification. Affinity-enhancing modifications and mutagenic modifications can be introduced in any nucleotide residue, and can be introduced at any position within a nucleotide residue, including positions on the base and on the ribose group. For example, pyrimidine bases can be readily modified at the 5 position, and nucleoside and nucleotide ribose groups can be readily modified at the 2′, 3′, and 5′ positions.

Examples of oligonucleotides, particularly TFOs, modified with one or more cationic functional groups are disclosed herein. Cationic functional groups, such as amino groups, can enhance the affinity of a modified oligonucleotide for a specific DNA sequence. One type of modified oligonucleotide comprises a 2′-O-alkylated residue. Such residues are particularly useful in TFOs, therefore, in one embodiment, a TFO includes one or more 2′-aminoethoxy ribose derivative. The amino group is protonated at physiological pH and thus the 2′-aminoethoxy group is a cationic functional group. In specific embodiments, the 2′-O-alkylated residues are pyrimidine residues, and in certain examples of modified oligonucleotides solely pyrimidine residues are 2′-O-alkylated. Modified residues can be in a region of the nucleotide. For example, two or more modified residues can be localized within six residues of one another, and in certain examples two or more residues are contiguous. In particular embodiments, oligonucleotides including three or four contiguous modified residues, particularly where the three or four contiguous residues are at the 3′ or 5′ terminus, are particularly active in vivo. In other particular embodiments, a sequence of three or four contiguous modified residues begins one or two residues away from the 3′ or 5′ terminus of the oligonucleotide.

Typically the modified oligonucleotides have from 5 to about 100 residues, more typically have from about 10 to about 50 residues, and even more typically from about 10 to about 25 residues. DNA modifying modified oligonucleotide sequences can include polypyrimidine motifs, polypurine motifs or both. In one embodiment modified oligonucleotides of between 10 and 25 residues include no more than four 2′-O-alkylated residues.

Embodiments of the disclosed methods relate to methods for mutating or provoking mutation in a target nucleotide sequence in a cellular genome using TFOs as targeting agents. The mutation can be induced by a mutagen attached to a TFO or by a TFO having sufficient affinity for the target sequence to render DNA repair error-prone. The mutation can be any of several types, and can be induced in sequence specific fashion. For example, the nucleotide sequence can be specifically targeted by the TFO, thus enabling targeted mutagenesis.

The disclosure also relates to cells, tissues, and organisms that have been modified by the disclosed methods and/or DNA modifying molecules. Cells from any animal can be modified, with particular examples including, human, mouse, hamster, sheep, pig, rabbit and cow. In particular embodiments modified cells include those selected from the group consisting of a blastomere cell, an eight-cell embryo cell, a blastocoele cell, a midgestation embryo cell or an embryonic stem cell. In one embodiment, DNA modification is enhanced when the cell is DNA repair-deficient.

Additional advantages will be set forth in part in the description which follows, and in part will be apparent from the description, or can be learned by practice disclosed methods. The advantages of the oligonucleotides and methods disclosed herein will be realized and attained by means of the elements and combinations particularly pointed out in the appended claims. It is to be understood that both the foregoing general description and the following detailed description are exemplary and explanatory only and are not restrictive.

### BRIEF DESCRIPTION OF THE DRAWINGS

FIG. 1A shows the sequence of the HPRT 14/E5 target (duplex sequence at top) and the modified oligonucleotides with varying patterns of 2′-AE substitution. The target is in Intron 4 adjacent to Exon 5 (the exon sequence is shown in lower case letters). The targeted psoralen crosslink site (TA) is shown in larger, upper-case typeface in the target duplex sequence. The modified oligonucleotides are parallel to the purine strand in the triplex. M AE-06 has a single mismatch (the underlined C). FIG. 1B shows the structures of the 2′ substituted (2′-OMe and 2′-AE), 5-methyl cytidine (C) and 5-methyl uridine (T).

FIG. 2A shows the absorbance at 254 nm versus temperature showing triplex and duplex melts of the deoxynucleotide analog of PS-01 (dot) and Ps-01 (solid) with 19-mer duplex. A two-step transition [1^{st} transition: Hoogsteen base pair (bp) melt, 2^{nd} transition: Watson-Crick bp melt] is observed for the deoxynucleotide analog of PS-01 (dot), while a one step change [single transition: combined Hoogsteen bp and Watson-Crick bp melt] is seen for PS-01 (solid). FIG. 2B shows the absorbance at 254 nm versus temperature showing the increments in the single transition melts of the complexes of PS-01 (solid), AE-04 (dash), AE-05 (dot), AE-06 (dash dot) and AE-07 (dash dot dot) with the 19-mer duplex target (Methods).

FIG. 3A shows the melting curve of triplexes of PS-01 with 19-mer duplex at pH 6.0 (solid), pH 6.5 (dash), pH 7.0 (dot) and pH 7.5 (dash dot). FIG. 3b shows the melting curve of triplexes of AE-06 with a 19-mer duplex target (Methods and Materials) at pH 6.0 (solid), pH 6.5 (dash), pH 7.0 (dot) and pH 7.5 (dash dot).

FIG. 4 shows the graphic representation of the drop in Tm as a function of pH, from pH 6.0 to pH 7.5, for PS-01 (square), AE-04 (circle), AE-05 (triangle), AE-06 (open square) and AE-07 (open circle).

FIG. 5A shows the binding isotherms of PS-01 (open triangles) and AE-06 (filled triangles) in 1 mM MgCl₂. FIG. 5B shows modified oligonucleotide K_{D} at 10 mM MgCl₂. The Hill's coefficients were: PS-01: 0.95; AE-04: 1.63; AE-05: 1.53; AE-06: 1.82; AE-07: 1.01. FIG. 5c shows modified oligonucleotide K_{D} at 1 mM MgCl₂. The Hill's coefficients were: PS-01: 1.35; AE-04: 1.84; AE-05: 1.37; AE-06: 2.43; AE-07: 1.97. The values of K_{D} are in the nanomolar scale.

FIG. 6 shows the stability of preformed triplexes in the cellular replication and mutagenesis compartment. Triplexes were preformed on psupF12 with PS-01 (squares) and AE-06 (circles) and introduced into Cos 1 cells by electroporation. At the indicated times, the cells were exposed to UVA light to photoactivate the psoralen. After an additional 48 hrs, the replicated plasmids were harvested and screened for mutations in the supF12 gene in a microbiological screen. Three independent experiments were performed with each modified oligonucleotide and the data points represent the mean and the standard error of the mean.

FIGS. 7A and C show the frequency of HPRT deficient cells following treatment with pso-modified oligonucleotides. Modified oligonucleotides were electroporated into CHO cells and the cells were treated with UVA 3 hrs later. The cells were carried for 8-10 days and then placed in thioguanine selection medium, while companion cultures were placed in non selective medium to score plating efficiency. The results are expressed as the percent of cells that were thioguanine resistant, corrected for plating efficiency. The mean and standard errors of the mean were: PS-01, 0.004±0.0008; AE-04, 0.005±0.001; AE-05, 0.01±0.005; AE-06, 0.11±0.01; AE-07, 0.14±0.04; AE-08, 0.05±0.01. The results for AE-06 represent 9 independent determinations, while 7 independent experiments were performed with AE-07 and AE-08.

FIG. 7B illustrates the lack of activity of the modified oligonucleotides in CHO cells against the APRT gene. Cells were placed in aza-adenine to select for cells deficient in APRT.

FIG. 8A shows the fluorescence-activated cell sorter profile of G/G cells, and FIG. 8B shows mid-S phase cells 4 hours after release from mimosine block.

FIG. 9 illustrates the observed frequency of Hprt⁻ cell colonies after treatment with AE-07 at different stages of the cell cycle (quiescent cells, G; 4 hours after release, G; in mimosine block, early S; 4 hours after release from mimosine, late S; untreated cells, control).

FIG. 10 illustrates the observed frequency of mutant colonies following treatment with free psoralen and UVA at the various stages of the cell cycle.

FIG. 11 depicts the results of XbaI digestion of PCR fragments of the target region from non-selected colonies of cells treated with AE-07/UVA in S-phase. The arrow indicates the undigested fragment.

FIG. 12 illustrates the results of a denaturation resistance assay of targeted crosslinks: lane 1 contained genomic DNA cross-linked in vitro to AE-07; lane 2 contained non-crosslinked DNA; lane 3 includes an equal mixture of the contents of lanes 1 and 2; lane 4 contained non-crosslinked, non-denatured DNA; lane C contained denatured DNA from untreated control cells; lanes Go and S contained denatured DNA from AE-07-treated G/G or S phase cells (two independent experiments). The arrowhead marks the position of the denatured fragment, and the arrow marks the non-denatured or denaturation-resistant fragment.

FIG. 13 illustrates hybridization with a probe to a 3-kb (arrow) fragment of the DHFR gene (samples not denatured).

FIG. 14 illustrates a blot of EcoRI and XbaI digestion of samples from AE-07 treated cells, control DNA digested with EcoRI (C) or EcoRI and XbaI (S or C), DNA from AE-07/UVA G/G or S phase cells digested with EcoRI and XbaI, respectively (the arrow marks the position of the XbaI-resistant fragment.

### SEQUENCE LISTING

SEQ ID NO: 1 is a target DNA oligonucleotide sequence used for thermal denaturation studies.

SEQ ID NO: 2 is a target DNA sequence used for TFO-DNA binding studies.

SEQ ID NO: 3 is a TFO (PS-01) comprising seventeen 2′-O-methyl ribose residues.

SEQ ID NO: 4 is a TFO (AE-04) comprising a single 2′-aminoethoxy residue and sixteen 2′-O-methyl ribose residues.

SEQ ID NO: 5 is a TFO (AE-05) comprising two contiguous 2′-aminoethoxy residues and fifteen 2′-O-methyl ribose residues.

SEQ ID NO: 6 is a TFO (AE-06) comprising three adjacent 2′-aminoethoxy residues and fourteen 2′-O-methyl ribose residues.

SEQ ID NO: 7 is a TFO (AE-07) comprising four contiguous 2′-aminoethoxy residues and thirteen 2′-O-methyl ribose residues.

SEQ ID NO: 8 is a TFO (AE-08) comprising three contiguous, 3′-terminal, 2′-aminoethoxy residues and fourteen 2′-O-methyl ribose residues.

SEQ ID NO: 9 is a TFO (M AE-06) comprising three contiguous 2′-aminoethoxy residues and fourteen 2′-O-methyl ribose residues, and having one mismatched base relative to a target sequence.

SEQ ID NO: 10 is a TFO (AE-18) comprising three non-contiguous 2′-aminoethoxy residues and fourteen 2′-O-methyl ribose residues.

SEQ ID NO: 11 is a TFO (AE-31) comprising three 2′-aminoethoxy ribose residues and fourteen 2′-O-methyl ribose residues.

SEQ ID NO: 12 is a TFO (AE-32) having three contiguous, 5′ terminal 2′-aminoethoxy ribose residues and fourteen 2′-O-methyl ribose residues.

SEQ ID NO: 13 is a TFO (AE-02) having six 2′-aminoethoxy ribose residues and eleven 2′-O-methyl ribose residues.

### DETAILED DESCRIPTION

The following explanations of terms and methods are provided to better describe the present compounds, compositions and methods and to guide those of ordinary skill in the art in the practice of the present disclosure. It is also to be understood that the terminology used in the disclosure is for the purpose of describing particular embodiments and examples only and is not intended to be limiting.

As used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint.

In this specification and in the claims which follow, reference will be made to a number of terms which shall be defined to have the following meanings:

“Optional” or “optionally” means that the subsequently described event or circumstance can or cannot occur, and that the description includes instances where said event or circumstance occurs and instances where it does not.

Variables such as A, V, W, X, Y, Z, and R¹-R⁵ used throughout the application are the same variables as previously defined unless stated to the contrary.

“Derivative” refers to a compound or portion of a compound that is derived from or is theoretically derivable from a parent compound.

The term “alkyl group” is defined as a branched or unbranched saturated hydrocarbon group of 1 to 24 carbon atoms, such as methyl, ethyl, n-propyl, isopropyl, n-butyl, isobutyl, t-butyl, pentyl, hexyl, heptyl, octyl, decyl, tetradecyl, hexadecyl, eicosyl, tetracosyl and the like. A “lower alkyl” group is a saturated branched or unbranched hydrocarbon having from 1 to 10 carbon atoms.

The term “alkenyl group” is defined as a hydrocarbon group of 2 to 24 carbon atoms and structural formula containing at least one carbon-carbon double bond.

The term “alkynyl group” is defined as a hydrocarbon group of 2 to 24 carbon atoms and a structural formula containing at least one carbon-carbon triple bond.

The term “halogenated alkyl group” is defined as an alkyl group as defined above with one or more hydrogen atoms present on these groups substituted with a halogen (F, Cl, Br, I).

The term “cycloalkyl group” is defined as a non-aromatic carbon-based ring composed of at least three carbon atoms. Examples of cycloalkyl groups include, but are not limited to, cyclopropyl, cyclobutyl, cyclopentyl, cyclohexyl, etc. The term “heterocycloalkyl group” is a cycloalkyl group as defined above where at least one of the carbon atoms of the ring is substituted with a heteroatom such as, but not limited to, nitrogen, oxygen, sulfur, or phosphorous.

The term “aliphatic group” is defined as including alkyl, alkenyl, alkynyl, halogenated alkyl and cycloalkyl groups as defined above.

The term “aryl group” is defined as any carbon-based aromatic group including, but not limited to, benzene, naphthalene, etc. The term “aromatic” also includes “heteroaryl group,” which is defined as an aromatic group that has at least one heteroatom incorporated within the ring of the aromatic group. Examples of heteroatoms include, but are not limited to, nitrogen, oxygen, sulfur, and phosphorous. The aryl group can be substituted with one or more groups including, but not limited to, alkyl, alkynyl, alkenyl, aryl, halide, nitro, amino, ester, ketone, aldehyde, hydroxy, carboxylic acid, or alkoxy, or the aryl group can be unsubstituted.

The term “aralkyl” is defined as an aryl group having an alkyl group, as defined above, attached to the aryl group. An example of an aralkyl group is a benzyl group.

The term alkyl amino includes alkyl groups as defined above where at least one hydrogen atom is replaced with an amino group.

The term “hydroxyl group” is represented by the formula —OH. The term “alkoxy group” is represented by the formula —OR, where R can be an alkyl group, optionally substituted with an alkenyl, alkynyl, aryl, aralkyl, cycloalkyl, halogenated alkyl, or heterocycloalkyl group as described above.

The term “hydroxyalkyl group” is defined as an alkyl group that has at least one hydrogen atom substituted with a hydroxyl group. The term “alkoxyalkyl group” is defined as an alkyl group that has at least one hydrogen atom substituted with an alkoxy group described above. Where applicable, the alkyl portion of a hydroxyalkyl group or an alkoxyalkyl group can have aryl, aralkyl, halogen, hydroxy, alkoxy

The term “amine group” is represented by the formula —NRR′, where R and R′ can be, independently, hydrogen or an alkyl, alkenyl, alkynyl, aryl, aralkyl, cycloalkyl, halogenated alkyl, or heterocycloalkyl group described above.

The term “amide group” is represented by the formula —C(O)NRR′, where R and R′ independently can be a hydrogen, alkyl, alkenyl, alkynyl, aryl, aralkyl, cycloalkyl, halogenated alkyl, or heterocycloalkyl group described above.

The term “ester” is represented by the formula —OC(O)R, where R can be an alkyl, alkenyl, alkynyl, aryl, aralkyl, cycloalkyl, halogenated alkyl, or heterocycloalkyl group described above.

The term “carbonate group” is represented by the formula —OC(O)OR, where R can be an alkyl, alkenyl, alkynyl, aryl, aralkyl, cycloalkyl, halogenated alkyl, or heterocycloalkyl group described above.

The term “carboxylic acid” is represented by the formula —C(O)OH.

The term “aldehyde” is represented by the formula —C(O)H.

The term “keto group” is represented by the formula —C(O)R, where R is an alkyl, alkenyl, alkynyl, aryl, aralkyl, cycloalkyl, halogenated alkyl, or heterocycloalkyl group described above.

The term “carbonyl group” is represented by the formula C═O.

The term “ether group” is represented by the formula R(O)R′, where R and R′ can be, independently, an alkyl, alkenyl, alkynyl, aryl, aralkyl, cycloalkyl, halogenated alkyl, or heterocycloalkyl group described above.

The term “halide” is defined as F, Cl, Br, or I.

The term “urethane” is represented by the formula —OC(O)NRR′, where R and R′ can be, independently, hydrogen, an alkyl, alkenyl, alkynyl, aryl, aralkyl, cycloalkyl, halogenated alkyl, or heterocycloalkyl group described above.

The term “hydrocarbon chain” refers to a chain of carbon atoms, typically comprising from 2 to about 20 carbon atoms. The chain can comprise aliphatic and aryl groups and can comprise straight chain, branched chain and/or cyclic groups.

The term “polyalkylene oxide” can be represented by the formula [—(CH₂)ₘ—O]ₙ—, where m and n independently are integers from 1 to about 10 and from 2 to about 20, respectively.

The term “polyalkylene imine” can be represented by the formula [—(CH₂)ₘ—NR]ₙ— where R is H or alkyl, and m and n independently are integers from 2 to about 10 and from 2 to about 20, respectively.

The term “silyl group” is represented by the formula —SiRR′R″, where R, R′, and R″ can be, independently, an alkyl, alkenyl, alkynyl, aryl, aralkyl, cycloalkyl, halogenated alkyl, alkoxy, or heterocycloalkyl group described above.

The terms “pyrimidine” and “purine” refer to nucleic acid bases derived from the heterocyclic pyrimidine and purine ring systems shown below:

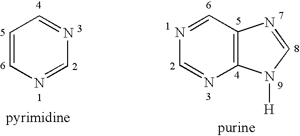

Pyrimidine and purine bases suitable for incorporation into oligonucleotides include substituted pyrimidines, such as cytosine, uracil, thymine, guanine, adenine, and derivatives thereof, and substituted purines, such as, guanine, adenine and derivatives thereof. Additional pyrimidine and purine derived bases, and bases derived from different ring systems are disclosed throughout the specification.

The groups described above can be optionally substituted with one or more substituents. The definition of any substituent or variable at a particular location in a molecule is independent of its definitions elsewhere in that molecule. Examples of suitable substituents include but are not limited to alkyl, alkenyl, alkynyl, aryl, halo, trifluoromethyl, trifluoromethoxy, hydroxy, alkoxy, oxo, alkanoyl, alkanoyloxy, aryloxy, amino, amido, alkanoylamino, aroylamino, aralkanoylamino, substituted alkanoylamino, aroylamino, aralkanoylamino, substituted alkanoylamino, substituted arylamino, substituted aralkanoylamino, thiol, sulfide, thiono, sulfonyl, sulfonamide, nitro, cyano, carboxy, carbamyl, substituted carbamyl and the like.

It is understood that substituents and substitution patterns of the compounds described herein can be selected by one of ordinary skill in the art to provide compounds that are chemically stable and that can be readily synthesized by techniques known in the art and further by the methods set forth in this disclosure.

With respect to formula I (shown below), Z and R¹-R⁵ can, independently, possess two or more of the groups listed above. For example, if R¹ is a straight chain alkyl group, one of the hydrogen atoms of the alkyl group can be substituted with a hydroxyl group, an alkoxy group, etc. Depending upon the groups that are selected, a first group may be incorporated within second group or, alternatively, the first group may be pendant or attached to the second group. For example, with the phrase “an alkyl group comprising an ester group,” the ester group may be incorporated within the backbone of alkyl group. Alternatively, the ester can be attached the backbone of the alkyl group. The nature of the group(s) that is (are) selected will determine if the first group is embedded or attached to the second group.

The term “modified oligonucleotide” is defined herein as an oligonucleotide that includes at least one modification, such as, without limitation, a base other than thymine, cytosine, uridine, adenine and guanine, a modified ribose residue, for example, a fluorinated ribose derivative, or a ribose derivative having an alkylated or acylated hydroxyl group, a modified backbone, for example, a phosphoramidate or phosphorothioate backbone. Additional modifications present in modified oligonucleotides are described throughout the disclosure.

The term “mutation” refers to a deletion, insertion, substitution, strand break, and/or adduct introduction. A “deletion” is defined as a change in a nucleic acid sequence in which one or more nucleotides is absent. An “insertion” or “addition” is that change in a nucleic acid sequence that has resulted in the addition of one or more nucleotides. A “substitution” results from the replacement of one or more nucleotides by a molecule which is a different molecule from the replaced one or more nucleotides. For example, a nucleic acid can be replaced by a different nucleic acid as exemplified by replacement of a thymine by a cytosine, adenine, guanine, or uridine. Alternatively, a nucleic acid can be replaced by a modified nucleic acid as exemplified by replacement of a thymine by thymine glycol. The term “strand break” when made in reference to a double stranded nucleic acid sequence includes a single-strand break and/or a double-strand break. A single-strand break refers to an interruption in one of the two strands of the double stranded nucleic acid sequence. This is in contrast to a double-strand break, which refers to an interruption in both strands of the double stranded nucleic acid sequence. Strand breaks can be introduced into a double stranded nucleic acid sequence either directly, for example, by ionizing radiation or indirectly, for example, by enzymatic incision at a nucleic acid base. A mismatch of DNA sequences can result in a mutation. The term “mismatch” refers to a non covalent interaction between two nucleic acids, each nucleic acid residing on a different polynucleic acid sequence, which does not follow the base-pairing rules. For example, for the partially complementary sequences 5-AGT-3′ and 5′-AAT-3′, a G-A mismatch is present. The terms “introduction of an adduct” or “adduct formation” refer to the covalent or non-covalent linkage of a molecule to one or more nucleotides in a DNA sequence such that the linkage results in a reduction (from 10% to 100%, from 50% to 100%, or from 75% to 100%) in the level of DNA replication and/or transcription.

The terms “mutant cell” and “modified cell” refer to a cell which contains at least one mutation in the cell's genomic sequence.

The term “cell” refers to a single cell. The term “cells” refers to a population of cells. The population can be a pure population comprising one cell type. Likewise, the population can comprise more than one cell type. There is no limit on the number of cell types that a cell population can comprise.

The term “synchronize” or “synchronized,” when referring to a sample of cells, or “synchronized cells” or “synchronized cell population” refers to a plurality of cells that have been treated to cause the population of cells to be in the same phase of the cell cycle. It is not necessary that all of the cells in the sample be synchronized. A small percentage of cells can be unsynchronized with the majority of the cells in the sample. A preferred range of cells that are synchronized is between 50-100%. A more preferred range is between 75-100%. Also, it is not necessary that the cells be a pure population of a single cell type. More than one cell type can be contained in the sample. In this regard, only one of cell types can be synchronized or can be in a different phase of the cell cycle as compared to another cell type in the sample.

The term “cell cycle” refers to the physiological and morphological progression of changes that cells undergo when dividing (proliferating). The cell cycle is generally recognized to be composed of phases termed “interphase,” “prophase,” “metaphase,” “anaphase,” and “telophase.” Additionally, parts of the cell cycle can be termed “M (mitosis),” “S (synthesis),” “G0,” G1 (gap 1)” and “G2 (gap 2).” Furthermore, the cell cycle includes periods of progression that are subdivisions of the above named phases, for example, early, mid-, and late S phase. Early, mid-, and late S phase are defined, for example, with respect to the total time required for the completion of S phase. Early S-phase is the first 20% of S phase, mid-S phase is from 20-40% through S phase, and late S phase is the last 40% to 100% of the time the cell spends in S phase. In particular embodiments, mid- to late S phase is the last 20-100% of S phase, and in particular embodiments is the last 40-100%, for example 60-100% of S-phase. In working embodiments cells occupied S phase for about 6 to 8 hours, thus cells are defined as being in early S phase from 0 to about 2 hours after S phase initiation, in mid-S phase from about 2 to about 4 hours, and in late S phase at from about 4 to about 8 hours after S phase initiation.

The term “cell cycle inhibition” refers to the cessation of cell cycle progression in a cell or population of cells. Cell cycle inhibition is usually induced by exposure of the cells to an agent (chemical, proteinaceous or otherwise) that interferes with aspects of cell physiology to prevent continuation of the cell cycle.

“Proliferation” or “cell growth” refers to the ability of a parent cell to divide into two daughter cells repeatably thereby resulting in a total increase of cells in the population. The cell population can be in an organism or in a culture apparatus.

The term “capable of modifying DNA” or “DNA modifying means” refers to procedures, as well as endogenous or exogenous agents or reagents that have the ability to induce, or can aid in the induction of, changes to the nucleotide sequence of a targeted segment of DNA. Such changes can be made by the deletion, addition or substitution of one or more bases on the targeted DNA segment. It is not necessary that the DNA sequence changes confer functional changes to any gene encoded by the targeted sequence. Furthermore, it is not necessary that changes to the DNA be made to any particular portion or percentage of the cells.

The term “target sequence” refers to any nucleotide sequence, the manipulation of which can be deemed desirable for any reason, by one of ordinary skill in the art. Such nucleotide sequences include, but are not limited to, coding sequences of structural genes, for example, reporter genes, selection marker genes, oncogenes, drug resistance genes, growth factors, etc., and non-coding regulatory sequences that do not encode an mRNA or protein product, for example, promoter sequence, enhancer sequence, polyadenylation sequence, termination sequence, etc.

The term “gene” means the deoxyribonucleotide sequences comprising the coding region of a structural gene. A “gene” can also include non-translated sequences located adjacent to the coding region on both the 5′ and 3′ ends such that the gene corresponds to the length of the full-length mRNA. The sequences that are located 5′ of the coding region and present on the mRNA are referred to as 5′ non-translated sequences. The sequences that are located 3′ or downstream of the coding region and present on the mRNA are referred to as 3′ non-translated sequences. The term “gene” encompasses both cDNA and genomic forms of a gene. A genomic form or clone of a gene contains the coding region interrupted with non-coding sequences termed “introns” or “intervening regions” or “intervening sequences.” Introns are segments of a gene which are transcribed into heterogenous nuclear RNA (hnRNA); introns can contain regulatory elements such as enhancers. Introns are removed or “spliced out” from the nuclear or primary transcript; introns therefore are absent in the messenger RNA (mRNA) transcript. The mRNA functions during translation to specify the sequence or order of amino acids in a nascent polypeptide. In addition to containing introns, genomic forms of a gene can also include sequences located on both the 5′ and 3′ end of the sequences which are present on the RNA transcript. These sequences are referred to as “flanking” sequences or regions (these flanking sequences are located 5′ or 3′ to the non-translated sequences present on the mRNA transcript). The 5′ flanking region can contain regulatory sequences such as promoters and enhancers which control or influence the transcription of the gene. The 3′ flanking region can contain sequences that direct the termination of transcription, post transcriptional cleavage and polyadenylation.

A “purine-rich sequence” or a “polypurine sequence” in reference to a nucleotide sequence on one of the strands of a double-helical nucleic acid sequence is defined as a contiguous sequence of nucleotides wherein greater than 50% of the nucleotides of the target sequence contain a purine base. However, it is preferred that the purine-rich target sequence contain greater than 60% purine nucleotides, for example greater than 75% purine nucleotides, for example greater than 90% purine nucleotides and most preferably 100% purine nucleotides.

A “non-human animal” refers to any animal which is not a human and includes vertebrates such as rodents, non-human primates, ovines, bovines, ruminants, lagomorphs, porcines, caprines, equines, canines, felines, aves, etc. Preferred non-human animals are selected from the order Rodentia. “Non-human animal” additionally refers to amphibians, for example Xenopus, reptiles, insects, for example Drosophila and other non-mammalian animal species.

A “transgenic animal” refers to an animal that includes a transgene that is inserted into a cell and becomes integrated into the genome either of somatic and/or germ line cells of the animal. A “transgene” means a DNA sequence that is partly or entirely heterologous (not present in nature) to the animal in which it is found, or that is homologous to an endogenous sequence (a sequence that is found in the animal in nature) and is inserted into the animal's genome at a location which differs from that of the naturally occurring sequence. Transgenic animals that include one or more transgenes are within the scope of the disclosure. Additionally, a “transgenic animal” as used herein refers to an animal that has had one or more genes modified and/or “knocked out” (a mutation that renders the gene non-functional or made to function at reduced level is called a “knockout” mutation) by the methods herein, by homologous recombination, modified oligonucleotide mutation or by similar processes.

“Patient” is defined as a human or other animal, such as a mouse, dog, cat, horse, bovine or ovine and the like, that can be in need of alleviation or amelioration from a recognized medical condition. A “host” is defined as an animal or cell line (animal, plant or prokaryote) that can be used as a recipient for exogenous reagents and substances. In present context, for example, a host non-human zygote can be used to generate an animal that has a gene knockout mutation, has altered expression of a protein or has increased expression of a protein. Animals with one or more of these types of mutations can have commercial value.

A “transformed cell” is a cell or cell line that has acquired the ability to grow in cell culture for multiple generations, the ability to grow in soft agar, and/or the ability to not have cell growth inhibited by cell-to-cell contact. In this regard, transformation refers to the introduction of foreign genetic material into a cell or organism. Transformation can be accomplished by any method known which permits the successful introduction of nucleic acids into cells and which results in the expression of the introduced nucleic acid. “Transformation” includes but is not limited to such methods as transduction, transfection, microinjection, electroporation, and lipofection (liposome-mediated gene transfer). Transformation can be accomplished through use of any expression vector. For example, the use of baculovirus to introduce foreign nucleic acid into insect cells is contemplated. The term “transformation” also includes methods such as P-element mediated germline transformation of whole insects. Additionally, transformation refers to cells that have been transformed naturally, usually through genetic mutation.

Reference will now be made in detail to the present preferred embodiments. Some examples of which are illustrated in the accompanying drawings. Whenever possible, the same reference numbers are used throughout the drawings to refer to the same or like parts.

### I. DNA Modifying Molecules

DNA modifying molecules as disclosed herein include, mutagens, DNA targeting agents, and molecules that include both a mutagen and a DNA targeting agent.

A. Mutagens

Mutagens can be used to provoke a desired modification or mutation in a target DNA sequence. Examples of useful mutagens include, without limitation, indolocarbazoles, napthalene diimide (NDI), transplatin, bleomycin, analogs of cyclopropapyrroloindole, phenanthodihydrodioxins, chlorambucil, mitomycin derivatives, enediyne derivatives, such as lidamycin, esperamicin, calicheamicin, dynemycin and the like, hematoporphyrin derivatives, coumarin derivatives, such as psoralen analogs, oxazolopyridocarbazole, daunomycine, anthraquinone, acridine orange, cis platinum analogs, radionuclides, such as ¹²⁵I, ³⁵S, ³²P, boron agents (which can be activated by neutron capture), and iodine (which can be activated with auger electrons).

The listed mutagen classes operate by various mechanisms, in particular, indolocarbazoles are topoisomerase I inhibitors. Inhibition of topoisomerase enzymes results in strand breaks and DNA protein adduct formation (Arimondo et al., Bioorg. Med. Chem. (2000) 8:777). NDI is a photooxidant that can oxidize guanines, which can cause mutations at sites of guanine residues (Nunez, et al., Biochemistry (2000) 39:6190). Bleomycin, for example, functions as a DNA breaker, and has been used a radiation mimetic.

Several preferred mutagens, including particular mutagenic groups described above are intercalators. Lerman first described intercalation as the insertion of a flat, aromatic chromophore between adjacent base pairs of the double helix. (Lerman, L. S. (1961) J. Mol. Biol. 3:18-30). In general, intercalator is a term that refers to any group that inserts between stacked bases. Intercalators include molecules which are potent antibiotic and antitumor drugs. (Neidle and Abraham, CRC Crit. Rev. Biochem. (1984)17:73-121; Wang, A. H-J. Curr. Opin. Struct. Biol. (1992) 2:361-368). Numerous intercalators suitable for inducing mutations are known to those of ordinary skill in the art. Psoralen is an example of a group that is both an intercalator and a reactive group that induces mutation in DNA sequences. Psoralen can be photoactivated to undergo 2+2 cycloaddition with an alkene group, such as a thymidine group.

B. DNA Targeting Agents

DNA targeting agents include any molecule that has an affinity for DNA, however preferred agents exhibit sequence selective or sequence specific affinity. Although a degree of sequence specificity between the targeting agent and the target duplex DNA, no particular degree of specificity is required, as long as a DNA-DNA targeting agent complex can form. Exemplary DNA targeting agents include those described below.

Peptide nucleic acid (PNA) molecules are one example of DNA targeting agents capable of sequence specific DNA recognition. PNAs are nucleic acids wherein the phosphate backbone is replaced with an N-aminoethylglycine-based polyamide structure. PNAs generally have a higher affinity for complementary nucleic acids than their natural counterparts following the Watson-Crick base-pairing rules. Moreover, PNAs can form highly stable triple helix structures with DNA. (See, e.g., U.S. Pat. No. 5,986,053, to Ecker, which is incorporated herein by reference).

Sequence specific zinc finger proteins can be used as DNA targeting agents. Moreover, zinc finger proteins can be selected and or rationally designed to target specific DNA sequences. See, Rebar et al., Science (1994) 263:671-673, and U.S. Pat. No. 6,534,261, to Cox et al., both publications are incorporated herein by reference.

Additional useful sequence specific DNA recognition and targeting agents include polyamides, such as pyrrole imidazole polyamides, as taught by U.S. Pat. No. 6,143,901 to Dervan et al., which is incorporated herein by reference.

The contributions of positive charge and an RNA-like sugar conformation have been combined in the 2′-O-(2-aminoethoxy) (2′-AE) ribose derivatives developed by Cuenoud and colleagues (39-41). TFOs carrying these substitutions show enhanced kinetics of triplex formation and greater stability of the resultant complex at physiological pH and low Mg⁺² concentration. NMR analysis indicates that the 2′-AE side chain occupies the gauche⁺ conformation, resulting in a specific interaction between the amine and the i-1 phosphate group in the purine strand of the duplex (42). Presently disclosed oligonucleotides exploit the benefits of 2′-AE ribose derivatives to give mutagenic TFOs having increased affinity for duplex DNA.

DNA targeting agents can induce a desired mutation by binding with sufficient affinity to provoke error-prone repair (Wang et al., Science (1996) 271:802-805). Alternatively, the DNA targeting agents can be tethered to a mutagen, thus providing a sequence selective DNA modifying molecule. Methods for tethering mutagens to DNA targeting agents are known to those of ordinary skill in the art. Exemplary methods for tethering mutagens to DNA targeting agents are disclosed by: Havre et al, in Proc. Nat. Acad. Sci., U.S.A. (1993) 90:7879-7883; Chan et al., J. Biol. Chem. (1999) 272:11541-11548; Bendinskas et al., Bioconjugate Chem. (1998) 9:555; Lukhtanov, et al, Nucleic Acids Res. (1997) 25:5077; all of which are incorporated herein by reference.

In one embodiment, mutagenic oligonucleotides are disclosed, where the mutagenic oligonucleotides have at least one unit according to formula I

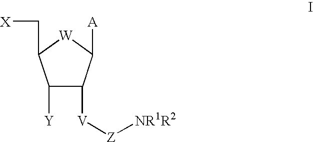

wherein A is a residue of a nucleic acid base;

X and Y are, independently, the same or different residues of an internucleosidic bridging group or a terminal group;

V and W are, independently, oxygen, sulfur, NR³, or CR⁴R⁵;

Z is an alkyl group, a cycloalkyl group, a heterocyloalkyl group, a hydroxyalkyl group, a halogenated alkyl group, an alkoxyalkyl group, an alkenyl group, an alkynyl group, an aryl group, a heteroaryl group, an aralkyl group, or a combination thereof;

R¹, R², R³, R⁴, and R⁵ are, independently, hydrogen, an alkyl group, a cycloalkyl group, a heterocyloalkyl group, an alkoxy group, a hydroxyalkyl group, a halogenated alkyl group, an alkoxyalkyl group, an alkenyl group, an alkynyl group, an aryl group, a heteroaryl group, an aralkyl group, a hydroxy group, an amine group, an amide, an ester, a carbonate group, a carboxylic acid, an aldehyde, a keto group, an ether group, a halide, a urethane group, a silyl group, or a combination thereof, wherein R¹ and R² can be part of a ring;

a salt thereof; and

a mutagen covalently attached to the mutagenic oligonucleotide.

The term “mutagenic oligonucleotide” is defined as an oligonucleotide comprising a unit according to formula I and at least one mutagenic group.

Mutagenic groups are defined as substituents that provoke or induce a mutation in a target DNA sequence. Typically the mutagenic group is covalently linked to the TFO so that the mutation occurs in a site-directed fashion. Certain mutagenic groups are DNA reactive (can react with a DNA sequence). DNA reactive groups include, without limitation, radionuclides, crosslinkers, alkylators, base modifiers, DNA breakers, free radical generators, combinations thereof, and other reagents. Reactive groups include any group that reacts with, can be induced to react with another group, or induces another group to form or cleave a covalent bond. For example, such DNA-reactive reagents release radicals which result in DNA strand breakage. Alternatively, the reagents alkylate DNA to form adducts that would block replication and transcription or induce error prone replication. In another alternative, the reagents generate crosslinks or molecules that inhibit cellular enzymes leading to strand breaks. In certain embodiments, the mutagenic group does not react with the target DNA sequence, yet still induces a desired mutation.

Examples of reactive and non-reactive mutagens that can be attached to a TFO include, those discussed above. For example, transplatin has been shown to react with DNA in a triplex target when the TFO is linked to the reagent. This reaction causes the formation of DNA adducts which breaks into duplex DNA on photoactivation (Bendinskas et al., Bioconjugate Chem. (1998) 9:555).

Mutagenic groups can be masked and/or can require activation to react with a target DNA sequence. Thus, such mutagenic groups attached to TFOs can be unmasked or activated to react at an optimum time, for example, after triplex formation and at the optimum stage of the cell cycle. This increases the desired mutation frequency and lowers the rate of non-selective mutation. As noted above, boron-containing molecules can be activated by neutron capture and iodine can be activated by auger electrons. Further activatable groups include photoactivatable groups, such as psoralen derivatives. Particular useful psoralen analogs are disclosed by Balbi et al. in Tetrahedron, 1994, 50, 4009-4020, which is incorporated herein by reference. Particularly reactive mutagens, such as enediynes, can be masked, for example by a method such as that disclosed by Takahishi et al. in Angew. Chem., Int. Ed. Engl. (1997) 36:1524-1526, which is incorporated herein by reference.

In embodiments where the mutagenic group is covalently attached to the modified oligonucleotide, the mutagenic group can be attached at any residue and at any position on nucleic acid residue. For example, the mutagenic group can be attached to a ribose residue at the 2′, 3′ or 5′ positions. If the mutagenic group is attached at the 3′ or 5′ position of a ribose residue, the residue typically is a terminal residue. Other sites for attachment of a mutagenic group include base functional groups. For example, the 5 position of pyrimidine residues can be readily functionalized. Moreover, pyrimidines can be derivatized at other positions, including the 4 position exocyclic amino group of cytosine. Purines can be derivatized at several positions, including, without limitation, the 3, 5 and 7 positions of guanine, which are readily alkylated. Furthermore, as is known to those of ordinary skill in the art, the mutagenic group can be attached directly to an oligonucleotide residue, or can be attached by a linker group. U.S. Pat. No. 6,136,601, to Meyer, et al., which is incorporated herein by reference, teaches useful linker groups (“linker arms”) and methods and positions for their attachment to oligonucleotides.

In one embodiment, a mutagenic oligonucleotide including at least one unit having formula I also includes at least a second nucleic acid analog. Such analogs include those having the modified bases described above, and include analogs having modified carbohydrate groups. For example, the 2′ oxygen can be alkylated, such as to give a 2′-O-methoxy modified ribose, a 2′-O-methoxyethyl or the like. Other modifications include deoxyribonucleotides, such as 2′-deoxy 2′-fluoro ribose derivatives.

In one embodiment of the disclosure, TFOs lacking a mutagen bind with sufficient affinity to provoke mutation of a target DNA sequence via error-prone repair. Nonetheless, such TFOs are not defined as being “mutagenic oligonucleotides.”

Surprisingly, as illustrated by the presently disclosed data, oligonucleotides having extensive substitution of 2′-AE modified residues for unmodified residues results in lower in vivo activity than oligonucleotides having four or fewer 2′-AE modified residues. Therefore, in one embodiment, modified oligonucleotides comprising no more than 4 units having the formula I are disclosed

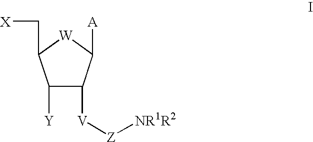

wherein A is a residue of a nucleic acid base;

X and Y are, independently, the same or different residues of an internucleosidic bridging group or a terminal group;

V and W are, independently, oxygen, sulfur, NR³, or CR⁴R⁵;

Z is an alkyl group, a cycloalkyl group, a heterocyloalkyl group, a hydroxyalkyl group, a halogenated alkyl group, an alkoxyalkyl group, an alkenyl group, an alkynyl group, an aryl group, a heteroaryl group, an aralkyl group, or a combination thereof;

R¹, R², R³, R⁴, and R⁵ are, independently, hydrogen, an alkyl group, a cycloalkyl group, a heterocyloalkyl group, an alkoxy group, a hydroxyalkyl group, a halogenated alkyl group, an alkoxyalkyl group, an alkenyl group, an alkynyl group, an aryl group, a heteroaryl group, an aralkyl group, a hydroxy group, an amine group, an amide, an ester, a carbonate group, a carboxylic acid, an aldehyde, a keto group, an ether group, a halide, a urethane group, a silyl group, or a combination thereof, wherein R¹ and R² can be part of a ring; or

a salt thereof.

The nucleic acid base A can be any natural nucleic acid base and known analogs of natural nucleic acids or novel chemical structures that function as nucleosidic analogs. A nucleic acid analog is a nucleic acid that contains some type of modification. Modifications would include natural and synthetic modifications of A, C, G, and T/U as well as different purine or pyrimidine bases, such as uracil-5-yl (.psi.) and 2-aminoadenin-9-yl. A modified base includes but is not limited to modified pyrimidines, such as 5-methylcytosine (5-me-C), 5-hydroxymethyl cytosine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 5-halo particularly 5-bromo, 5-trifluoromethyl and other 5-substituted uracils and cytosines,

A modified base also includes, but is not limited to, modified purines, such as 8-halo, 8-amino, 8-thiol, 8-thioalkyl, 8-hydroxyl and other 8-substituted adenines and guanines, xanthine, hypoxanthin-9-yl (I), hypoxanthine, 2-aminoadenine, 6-methyl and 2-propyl alkyl derivatives of adenine and guanine, and other alkyl derivatives of adenine and guanine, such as 7-alkylpurines, particularly 7-methylguanine and 7-methyladenine, other modified bases include aza and deaza purine analogs, such as 8-azaguanine and 8-azaadenine, 7-deazaguanine and 7-deazaadenine and 3-deazaguanine and 3-deazaadenine. Additional base modifications can be found for example in U.S. Pat. No. 3,687,808, Englisch et al., Angew. Chemie, Int. Ed. (1991) 30:613, and Sanghvi, Y. S., Chapter 15, Antisense Research and Applications, pages 289-302, Crooke, S. T. and Lebleu, B. eds., CRC Press, 1993. Certain nucleotide analogs, such as 5-substituted pyrimidines, 6-azapyrimidines and N-2, N-6 and 0-6 substituted purines, including 2-aminopropyladenine, 5-propynyluracil and 5-propynylcytosine. 5-methylcytosine can increase the stability of duplex formation. Often time base modifications can be combined with for example a sugar modification, such as 2′-O-methoxyethyl, to achieve unique properties such as increased duplex stability. There are numerous United States patents such as U.S. Pat. Nos. 4,845,205; 5,130,302; 5,134,066; 5,175,273; 5,367,066; 5,432,272; 5,457,187; 5,459,255; 5,484,908; 5,502,177; 5,525,711; 5,552,540; 5,587,469; 5,594,121, 5,596,091; 5,614,617; and 5,681,941, which detail and describe a range of base modifications. Each of these patents is herein incorporated by reference. Additionally, the nucleic acids and analogs thereof disclosed in reference numbers 65-86, which are incorporated by reference herein in their entireties, can be used to prepare modified oligonucleotides and can be used according to the present methods.

In one embodiment, the nucleic acid residue A can be any purine or pyrimidine. In another embodiment, the nucleic acid residue A is a purine, such as xanthine, hypoxanthine, adenine, 2-aminoadenine, guanine, thioguanine, 2,6-diaminopurine, purine, 7-deazaadenine, 7-deazaguanine, isoguanine, 2-aminopurine, N,N-dimethylguanine and derivatives thereof. In another embodiment A is a pyrimidine, such as uridine, cytosine, thymine, 6-uracil, 5-methylcytosine, 5-propynyluracil, 5-fluorouracil, 5-propynylcytosine, 5-propynyluracil, 6-methyluracil, 4-thiouracil, 2-pyrimidone, bromouracil, or a functional equivalent thereof. In a further embodiment, A is another heterocyclic base analog, for example a pyridine derivative, such as aminopyridine. In another embodiment, the nucleic acid A can be 5-substituted cytosine or 5-substituted uridine. In this embodiment, cytosine or uridine can be substituted at the 5 position with any of the groups previously defined for R¹-R⁵. 5-halo and 5-propynyl pyrimidine derivatives are particularly useful for further functionalization. For example, the 5 position halogen or alkyne moiety can be used to attach a linker group or other functional group by various methods, as is known to those of ordinary skill in the art. Specifically, an organometallic reaction, such as a Castro-type coupling reaction can be used to functionalize 5-halo pyrimidines and a Heck-type coupling reaction can be used to functionalize 5-alkynyl pyrimidines.

In one embodiment, X and Y are, independently, the same or different residues of internucleosidic bridging group or a terminal group. Internucleosidic bridging groups and methods of preparing them and introducing them into nucleoside building blocks and modified oligonucleotides are known to those of ordinary skill in the art. The term “internucleosidic bridging group” is defined as a group that connects two nucleosides. Some of the internucleosidic bridging groups can exist in different tautomeric forms depending upon, for example, the solvent and the degree of ionization of ionizable groups. For example, the bridging group in a phosphorothioate [O—(P—SH)(═O)—O] can be tautomerized to [O—(P—OH)(═S)—O].

In one embodiment, X and Y can form a phosphodiester group, a phosphorothioate group, a phosphodithioate, a methylphosphonate group, a H-phosphonate group, a phosphoramidate group, a phosphotriester group, a sulfonate group, a sulfite group, a sulfoxide group, a sulfide group, a formacetal group, a thioformacetal group, a thioether group, a hydroxylamine group, a methylene(methylimino) group, a methyleneoxy(methylimino) group, or an amide group. In another embodiment, X and Y are residues which together form a phosphodiester, phosphorothioate, or an amide bond between adjacent nucleosides or nucleoside analogs or together form an analog of an internucleosidic bond.

The term “terminal group” refers to a group at the 5′-position of the 5′ terminal residue or at the 3′-position of the 3′-terminal residue. The terminal groups of an oligonucleotide may be the same or different and any terminal group can be used. In certain embodiments a terminal group can be a hydroxyl or a phosphate group. In another embodiment a terminal group comprises a functionalized hydroxyl, for example, an ester or ether, or a functionalized phosphate, for example a phosphate ester, such as a phosphodiester. The 3′ and 5′ terminal positions are particularly useful positions for incorporating additional functional groups into an oligonucleotide because these positions are generally selectively protected and/or deprotected during oligonucleotide synthesis. In one embodiment, a terminal group comprises a mutagen, intercalator or reactive group. In a working embodiment a terminal group includes a phosphodiester and a psoralen derivative. Various terminal groups and methods for preparing such groups are known to those of ordinary skill in the art.

Similarly, various X and Y groups can be incorporated into oligonucleotides as is known to those of ordinary skill in the art. For example, in one embodiment X or Y can be introduced as a phosphoramidite derivative. In a working embodiment, a psoralen derivatized phosphoramidite, 2-[4′-(hydroxymethyl)-4,5′,8-trimethylpsoralen]-hexyl-1-O-(2-cyanoethyl)-(N,N-diisopropyl)-phosphoramidite was used to introduce a psoralen derivative to a 5′-oxygen.

In one embodiment, Z is a branched or straight chain C₂-C₆ alkyl group. For example, Z can be ethylene, propylene, butylene, pentylene, hexylene, isopropylene, isobutylene, or neopentylene. In one embodiment, Z is ethylene —(CH₂CH₂)—.

In one embodiment, R¹ and R² are, independently, hydrogen, methyl, ethyl, propyl, vinyl, or phenyl. In another embodiment, R¹ and R² are both hydrogen. In an alternate embodiment, R¹ and R² can be part of a ring system. For example, R¹ and R² can form a piperidine or morpholino ring.

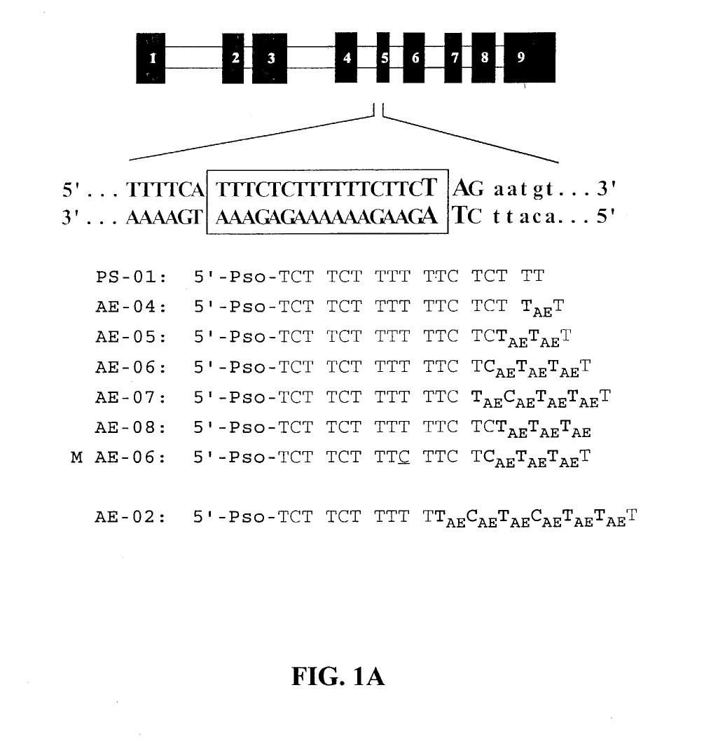

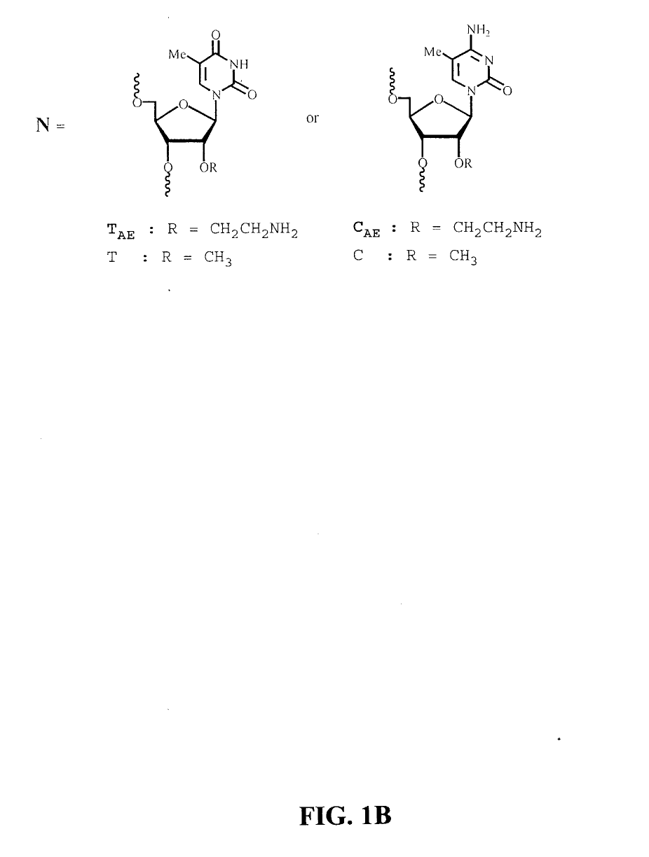

In one embodiment, the modified oligonucleotide has at least one unit having the formula I, wherein A is a residue of 5-methylcytosine or thymine; V and W are oxygen; X and Y together form a phosphodiester bond; Z is —CH₂CH₂—; and R¹ and R² are hydrogen. (FIG. 1b).

The modified oligonucleotides also encompass salts such as acidic salts, salts with bases, or if several salt-forming groups are present, mixed salts or internal salts. The salts are generally pharmaceutically-acceptable salts that are non-toxic.

In one embodiment, the modified oligonucleotides possess an acidic group. Examples of acidic groups include, but are not limited to, a carboxyl group, a phosphodiester group or a phosphorothioate group, that can form salts with suitable bases. These salts include, for example, nontoxic metal salts which are derived from metals of groups Ia, Ib, IIa and IIb of the Periodic Table of the elements. In one embodiment, alkali metal salts such as lithium, sodium or potassium salts, or alkaline earth metal salts such as magnesium or calcium salts can be used. The salt can also be zinc or an ammonium cation. The salt can also be formed with suitable organic amines, such as unsubstituted or hydroxyl-substituted mono-, di- or tri-alkylamines, in particular mono-, di- or tri-alkylamines, or with quaternary ammonium compounds, for example with N-methyl-N-ethylamine, diethylamine, triethylamine, mono-, bis- or tris-(2-hydroxy-lower alkyl)amines, such as mono-, bis- or tris-(2-hydroxyethyl)amine, 2-hydroxy-tert-butylamine or tris(hydroxymethyl)methylamine, N,N-di-lower alkyl-N-(hydroxy-lower alkyl)amines, such as N,N-dimethyl-N-(2-hydroxyethyl)amine or tri-(2-hydroxyethyl)amine, or N-methyl-D-glucamine, or quaternary ammonium compounds such as tetrabutylammonium salts.

In another embodiment, the modified oligonucleotides that possess a basic group that can form acid-base salts with inorganic acids. Examples of basic groups include, but are not limited to, an amino group or imino group. Examples of inorganic acids that can form salts with such basic groups include, but are not limited to, mineral acids such as hydrochloric acid, hydrobromic acid, sulfuric acid or phosphoric acid. Basic groups also can form salts with organic carboxylic acids, sulfonic acids, sulfo acids or phospho acids or N-substituted sulfamic acid, for example acetic acid, propionic acid, glycolic acid, succinic acid, maleic acid, hydroxymaleic acid, methylmaleic acid, fumaric acid, malic acid, tartaric acid, gluconic acid, glucaric acid, glucuronic acid, citric acid, benzoic acid, cinnamic acid, mandelic acid, salicylic acid, 4-aminosalicylic acid, 2-phenoxybenzoic acid, 2-acetoxybenzoic acid, embonic acid, nicotonic acid or isonicotonic acid, and, in addition, with amino acids, for example with α-amino acids, and also with methanesulfonic acid, ethanesulfonic acid, 2-hydroxymethanesulfonic acid, ethane-1,2-disulfonic acid, benzenedisulfonic acid, 4-methylbenzenesulfonic acid, naphthalene-2-sulfonic acid, 2- or 3-phosphoglycerate, glucose-6-phosphate or N-cyclohexylsulfamic acid (with formation of the cyclamates) or with other acidic organic compounds, such as ascorbic acid.

The modified oligonucleotides disclosed herein can be synthesized using techniques known in the art. The methods disclosed in “Oligonucleotide Synthesis, A Practical Approach” M. J. Gait; IRL Press 1984 (Oxford-Washington D.C.) and in U.S. Pat. No. 5,874,555 to Dervan et al., both which are incorporated by reference in their entireties, are useful for preparing modified oligonucleotides as well as conventional oligonucleotides. If necessary, functional groups of the nucleosides can be protected using techniques known in the art. In one embodiment, the modified oligonucleotide can be synthesized on a solid support. Linkers connecting the 3′ phosphate of a fragment of the oligonucleotides to the 5′-phosphate of the remaining fragment of the oligonucleotide also can be used in preparing the modified oligonucleotides. Such linkers include, but are not limited to, alkyl or aryl compounds. In another embodiment, the linkers can retain the three-carbon chain length, thereby approximating the interphosphate distance present in naturally occurring oligonucleotides. The linkers disclosed in reference numbers 87-96, which are incorporated herein by reference in their entireties, can be used.

The unit having formula I can be synthesized using techniques known in the art. For example, Cuenoud et al., Angew. Chem. Int. Ed. (1998) 37:1288-1291, discloses the synthesis 2′-aminoethoxy and 2′-aminopropoxy modified monomeric thymidine and C5-methylcytidine building blocks. By varying the starting materials, it is possible to use the procedure disclosed in Cuenoud et al. to make additional units, including 2′-aminoalkoxy modified residues, according to formula I.

Particular modified oligonucleotides disclosed herein have at least one unit having formula I. In one embodiment, the number of units having formula I is from 1 to 25, 1 to 10, 2 to 9, 3 to 8, 4 to 7, or 5 to 6. In another embodiment, the modified oligonucleotide has 3 or 4 units having formula I.

The location of the unit having formula I in the modified oligonucleotide can vary. In one embodiment, when the modified oligonucleotide has two or more units having formula I, the units are localized to a selected region of the oligonucleotide that is less than the entire length of the oligonucleotide. In one embodiment, the selected region of the modified oligonucleotide is less than 6 residues in length. In another embodiment, the selected region of the modified-oligonucleotide is 2, 3, 4, or 5 residues in length. In another embodiment, the units are adjacent to one another or separated by one or more naturally-occurring units or modified units not described by formula I. Specific examples have two or more groups of units having formula I that are localized at different regions of the modified oligonucleotide. In another embodiment, the units having formula I can be located in the middle of the oligonucleotide or at the 3′ or 5′ prime end of the oligonucleotide. In another embodiment, the modified oligonucleotide has 3 or 4 units having formula I adjacent to one another to form a patch. In working embodiments, modified oligonucleotides having consecutive units according to formula I had increased or enhanced bioactivity. Nonetheless, in some instances it is favorable for the oligonucleotide to have two or more units according to the formula dispersed throughout the oligonucleotide.

Modified oligonucleotides disclosed herein are capable of forming triple helical structures with the nucleic acid duplexes. “Triple helix” is generally defined as a double-helical nucleic acid with an oligonucleotide bound to a target sequence within the double-helical nucleic acid. The “double-helical” nucleic acid can be any double-stranded nucleic acid including double-stranded DNA, double-stranded RNA and mixed duplexes of DNA and RNA. The double-stranded nucleic acid is not limited to any particular length. In one embodiment, it has a length of greater than 500 bp, greater than 1 kb, or at most greater than about 5 kb. In many applications the double-helical nucleic acid is cellular, genomic nucleic acid. The modified oligonucleotide can bind to the target sequence in a parallel or anti-parallel manner.

The modified oligonucleotide is not limited to any particular length. In one embodiment, the length of the modified oligonucleotide is 200 nucleotides or less, 100 nucleotides or less, from 5 to 50 nucleotides, from 10 to 30 nucleotides, or from 15 to 25 nucleotides. Although a degree of sequence specificity between the modified oligonucleotide and the duplex DNA is necessary for formation of the triple helix, no particular degree of specificity is required, as long as the triple helix is capable of forming. Likewise, no specific degree of avidity or affinity between the modified oligonucleotide and the duplex helix is required as long as the triple helix is capable of forming.

In one embodiment, the localization of units having formula I to the selected region of the modified oligonucleotide increases nucleation of the modified oligonucleotide with nucleic acid duplexes to form a triple helical nucleus consisting of at least 3 to 5 base triplets. In another embodiment, the localization of units having the formula I to the selected region of the modified oligonucleotide increases the rate of nucleation (improved kinetics, increased rate of hybridization) between the modified oligonucleotide and the nucleic acid duplexes. In another embodiment, the localization of units having formula I to the selected region of the modified oligonucleotide decreases the rate at which the triple helical nucleus dissociates to form the modified oligonucleotide and the nucleic acid duplex.

In one embodiment, a vector comprises a modified oligonucleotide according to the present disclosure. In one embodiment, the vector is a nucleic acid vector or a viral vector.

Any of the modified oligonucleotides described herein can be combined with a pharmaceutically acceptable carrier to form a pharmaceutical composition.

Pharmaceutical carriers are known to those skilled in the art. These most typically would be standard carriers for administration of compositions to humans, including solutions such as sterile water, saline, and buffered solutions at physiological pH. The compositions could also be administered intramuscularly, subcutaneously, or in an aerosol form. Other compounds will be administered according to standard procedures used by those skilled in the art.

Molecules intended for pharmaceutical delivery can be formulated in a pharmaceutical composition. Pharmaceutical compositions can include carriers, thickeners, diluents, buffers, preservatives, surface active agents and the like in addition to the molecule of choice. Pharmaceutical compositions can also include one or more active ingredients such as antimicrobial agents, anti-inflammatory agents, anesthetics, and the like.

### II. Methods of Using DNA Modifying Molecules

The methods generally relate to using DNA modifying molecules to improve the efficiency of targeting mutations to specific locations in genomic or other nucleotide sequences. Additionally, the disclosure concerns target DNA that has been modified, mutated or marked using DNA modifying molecules as disclosed herein. One embodiment includes cells, tissue, and organisms that have been modified by any of the DNA modifying molecules disclosed herein. The disclosed DNA modifying molecules can be used in any of the methods disclosed in international publication no. WO 01/73001 A2, U.S. Pat. Nos. 5,776,744; 5,962,426; 6,303,376; 5,928,863; 5,693,471, U.S. application Ser. No. 08/473,845, and European patent no. 0705270, all of which are incorporated herein by reference in their entireties.

One disclosed embodiment relates to the discovery that the frequency of gene targeting can be substantially increased by treating the recipient cells during a specific window of time during the cell cycle (for example, during the DNA synthesis phase of the cell cycle). Thus a disclosed method allows targeting a mutation to an intracellular gene sequence at increased efficiency.

Thus, the method generally enables a method for the efficient mutation of genomic cellular DNA and/or recombination of DNA into the genomic DNA of cells. In one embodiment, the method exploits the novel observation that the mutation of genomic DNA is enhanced when the mutation event occurs in cell cycle-synchronized cells. In other words, cells have a higher frequency of mutation of their genomic DNA if the DNA modifying molecule is introduced into the cells during optimum points (phases) in the cell cycle. Therefore, a synchronized population of cells provides the greatest advantages in achieving the desired result of enhanced mutation of genomic DNA as long as the DNA mutation is performed during the optimal cell cycle period for that cell type and/or for the particular DNA modifying reagent which is used. For example, for a given cell type, the optimum phase of the cell cycle for modifying a target DNA can be different when using DNA modifying molecules that cause DNA strand breaks as compared to reagents which cause DNA alkylation or error-prone repair. Further details of this method are provided by PCT application number US01/09218, published Oct. 4, 2001, and incorporated herein by reference.

Although not limited to any particular use, the present methods are useful for introducing a mutation into the genome of a cell for the purpose of determining the effect of the mutation on the cell. In one embodiment, the mutation comprises deletion, insertion, substitution, strand break, adduct formation, gene conversion, or recombination of a novel sequence. For example, a mutation can be introduced into the nucleotide sequence that encodes an enzyme to determine whether the mutation alters the enzymatic activity of the enzyme, and/or determine the location of the enzyme's catalytic region. Alternatively, the mutation can be introduced into the coding sequence of a DNA-binding protein to determine whether the DNA binding activity of the protein is altered, and thus to delineate the particular DNA-binding region within the protein. Yet another alternative is to introduce a mutation into a non-coding regulatory sequence (for example, a promoter, enhancer, etc.) to determine the effect of the mutation on the level of expression of a second sequence that is operably linked to the non-coding regulatory sequence. In a preferred embodiment, the method accomplishes recombination or replacement of a defective gene with a functional gene. This can be desirable to, for example, define the particular sequence that possesses regulatory activity.

Target sequences can be determined for any gene sequence of interest including those found within databases such as GenBank (http://www.ncbi.nhn.nih.gov/PubMed/index.html). However, the target sequence can be contained in any gene for which the sequence is at least partially known. Target sequences within the gene sequence can be determined by the results desired. As mentioned above, the replacement, deletion or addition of specific sequences requires the determination of that particular sequence and the appropriate homologous sequence. For the embodiment in which inhibition of expression of a particular gene is involved, the selection of a sequence to which homology is needed need not be made with extreme precision. In one embodiment, when using modified oligonucleotides, triple helix formation is enhanced when the target region is a polypurine or homopurine region.

Illustrative genomic sequences which can be modified using the methods herein include, but are not limited to, sequences which encode enzymes; lymphokines, for example, interleukins, interferons, TNF, etc.; growth factors, for example, erythropoietin, G-CSF, M-CSF, GM-CSF, etc.; neurotransmitters or their precursors or enzymes responsible for synthesizing them; trophic factors, for example, BDNF, CNTF, NGF, IGF, GMF, aFGF, bFGF, NT3, NT5, HARP/pleiotrophin, etc.; apolipoproteins, for example, ApoAI, ApoAIV, ApoE. etc.; lipoprotein lipase (LPL); the tumor-suppressing genes, for example, p53, Rb, Rap1A, DCC k-rev, etc.; factors involved in blood coagulation, for example, Factor VII, Factor VIII, Factor IX, etc.; suicide genes, for example, thymidine kinase or cytosine deaminase; blood products; hormones; etc. In one embodiment, the genomic sequences encode enzymes and proteins which are involved in DNA repair. DNA repair enzymes and proteins are exemplified by, but not limited to, those which are involved in the nucleotide excision repair (NER) pathway, base excision repair (BER) pathway, double strand break, repair (DSBR) pathway, error prone polymerases, and enzymes which are involved in direct repair of DNA mutations.

In one embodiment, any of the cells treated with a DNA modifying molecule can be incorporated into an animal to ameliorate a medical condition. For example, a living cell of an animal can be contacted with a modified oligonucleotide to produce a modified cell. Genomic sequences have been associated with a human disease. Such genomic sequences are exemplified by, but not limited to, the adenosine deaminase (ADA) gene (GenBank Accession No. Ml 3792) associated with adenosine deaminase deficiency with severe combined immune deficiency; alpha-I-antitrypsin gene (GenBank Accession No. M11465) associated with alpha-I-antitrypsin deficiency; beta chain of hemoglobin gene (GenBank Accession No. NM000518) associated with beta thalassemia and Sickle cell disease; receptor for low density lipoprotein gene (GenBank Accession No. D116494) associated with familial hypercholesterolemia; lysosomal glucocerebrosidase gene (GenBank Accession No. K02920) associated with Gaucher disease; hypoxanthine guanine phosphoribosyltransferase (HPRT) gene (GenBank Accession No. M26434, J00205, M27558, M27559, M27560, M27561, M29753, M29754, M29755, M29756, M29757) associated with Lesch-Nyhan syndrome; lysosomal arylsulfatase A (ARSA) gene (GenBank Accession No. NM_{—}000487) associated with metachromatic leukodystrophy; omithine transcarbamylase (OTC) gene (GenBank Accession No. NM_{—}000531) associated with omithine transcarbamylase deficiency; phenylalanine hydroxylase (PAH) gene (GenBank Accession No. NM_{—}000277) associated with phenylketonuria; purine nucleoside phosphorylase (NP) gene (GenBank Accession No. NM_{—}000270) associated with purine nucleoside phosphorylase deficiency; the dystrophin gene (GenBank Accession Nos. M18533, M17154, and M18026) associated with muscular dystrophy; the utrophin (also called the dystrophin related protein) gene (GenBank Accession No. NM007124) whose protein product has been reported to be capable of functionally substituting for the dystrophin gene; and the human cystic fibrosis transmembrane conductance regulator (CFTR) gene (GenBank Accession No. M28668) associated with cystic fibrosis.

In another embodiment, the present methods can be used to generate transgenic cells and transgenic animals that are useful as models for diseases and for screening therapeutic reagents. For example, where a particular mutation to a gene in a first animal (for example, human) is known or thought to be associated with a disease, for example, lung cancer), the methods herein can be used to introduce the same or similar mutation into the genome of another animal (for example, mouse) to generate a transgenic animal which can be used as a model for the disease in the first animal. Transgenic animals can be generated using several methods that are known in the art, including microinjection, retroviral infection, and implantation of embryonic stem cells. For example, mutations can be introduced into fertilized eggs, cells from pre-implantation embryos such as blastomeres, eight-cell embryos, blastocoele, and midgestation embryos, and into embryonic stem (ES) cells.

Any type of cell which undergoes proliferation can be used according to the disclosed methods. Such cells are exemplified by embryonic cells (for example, oocytes, sperm cells, embryonic stem cells, 2-cell embryos, protocorm-like body cells, callus cells, 38 etc.), adult cells (for example, brain cells, fruit cells etc.), undifferentiated cells (for example, fetal cells, tumor cells, etc.), differentiated cells (for example, skin cells, liver cells, lung cells, breast cells, reproductive tract cells, neural cells, muscle cells, blood cells, T cells, B cells, etc.), dividing cells, senescing cells, cultured cells, and the like. Furthermore, the target cells can be primary cells or cultured cells. A “primary cell” is a cell which is directly obtained from a tissue or organ of an animal in the absence of culture. Preferably, though not necessarily, a primary cell is capable of undergoing ten or fewer passages in an in vitro culture before senescence and/or cessation of proliferation. In contrast, a “cultured cell” is a cell that has been maintained and/or propagated in vitro. Cultured cells include “cell lines”, such as cells that are capable of a greater number of passages in vitro before cessation of proliferation and/or senescence as compared to primary cells from the same source. One cell line includes cells that are capable of an infinite number of passages in culture.

In one embodiment, the cells are human and are exemplified by, but not limited to, U937 cells (macrophage), ATCC# crl 1593.2; A-375 cells (melalioma/melanocyte), ATCC# crl-1619; K LE cells (uterine endometrium), ATCC# crl-1622; T98G cells (glioblastoma), ATCC# crl-1690; CCF-STTG1 cells (astrocytoma), ATCC# erl-1718; HUV-EC-C cells (vascular endothelium), ATCC# CRL-1730; UM-UC-3 cells (bladder), ATCC# crl-1749; CCD841-CoN cells (colon, ATCC# crl-1790; SNU-423 cells (hepatocellular carcinoma), ATCC# crl-2238; W138 cells (lung, normal), ATCC# crl-75; Raji cells (lymphoblastoid), ATCC# ccl-86; BeWo cells (placenta, choriocarcinoma), ATCC# eel-98; HT1080 cells (fibrosarcoma), ATCC# ccl-121; MIA PaCa2 cells (pancreas), ATCC# crl-1420; CCD-25SK cells (skin fibroblast), ATCC# crl-1474; ZR75-30 cells (mammary gland), ATCC# crl-1504; HOS cells (bone osteosarcoma), ATCC# erl-1543; 293-SF cells (kidney), ATCC# crl-1573; LL47 (MaDo) cells (normal lymphoblast), ATCC# cc1-135; and HeLa cells (cervical carcinoma), ATCC# col-2.

In another embodiment, the cells are non-human and are exemplified by, but not limited to, LM cells (mouse fibroblast), ATCC# ccl-12; NCTC 3,526 cells' (rhesus monkey kidney), ATCC# ccl-7.2; BHK-21 cells (golden hamster kidney), ATCC# ccl-10; MD13K cells (bovine kidney), ATCC# ccl-22; PK 15 cells (pig kidney), ATCC# ccl-33; MI)CK cells (dog kidney), ATCC# ec1-34; PtK1 cells (kangaroo rat kidney), ATCC# ccl-35; Rk 13 cells (rabbit kidney), ATCC# ccl-37; Dede cells (Chinese hamster 39 lung fibroblast), ATCC# ccl-39; Bu (IMR31) cells (bison lung fibroblast), ATCC# ecl-40; FHM cells (minnow epithelial), ATCC# ecl-42; LC-540 cells (rat Leydig cell tumor), ATCC# eel-43; TH-1 cells (turtle heart epithelial), ATCC# ccl-SO; E. Derm (NBL-6) cells (horse fibroblast), ATCC# ecl-57; MvLn cells (mink epithelial), ATCC# ccl-64; Ch1 Es cells (goat fibroblast), ATCC# ccl-73; PI 1 Nt cells (raccoon fibroblast), ATCC# ccl-74; Sp I k cells (dolphin epithelial), ATCC# ccl-78; CRFK cells (cat epithelial), ATCC# ecl-94; Gekko Lung I cells (lizard-geldco epithelial), ATCC# ccl-111; Aedes aegypti cells (mosquito epithelial), ATCC# ccl-125; ICR 134 cells (frog epithelial), ATCC# ccl-128; Duck embryo cells (duck fibroblast), ATCC# ccl-141; and DBS Fcl-1 cells (monkey lung fibroblast), ATCC# ccl-161.

In an alternative embodiment, the cells are capable of generating an animal. Such cells are exemplified by, but not limited to, fertilized egg cells which can be implanted into the uterus of a pseudopregnant female and allowed to develop into an animal. These cells have successfully been used to produce transgenic mice, sheep, pigs, rabbits and cattle [Hammer et al., J. Animal Sci. (1986) 63:269; Hammer et al, Nature (1985) 315:680-683]. Other cells include pre-implantation embryo cells. For example, blastomere cells [Jaenisch, Proc. Natl. Acad. Sci. USA (1976) 73:1260-1264; [Jahner et al., Proc. Natl. Acad. Sci. USA (1985) 82:6927-6931; Van der Putten et al., Proc. Natl. Acad. Sci. USA (1985) 82:6148-6152], and eight-cell embryo cells from which the zona pellucida has been removed [Van der Putten (1985), supra; Stewart et al., EMBO J. (1987) 6:383-388]. The pre-implantation embryos which are manipulated in accordance with the methods herein can be transferred to foster mothers for continued development. Alternatively, cells can be at a later stage of embryonic development, such as blastocoele cells and midgestation embryo cells [Jahner et al., Nature (1982) 298:623 628]. Yet another cell type is an embryonic stem (ES) cell. ES cells are pluripotent cells that can be directly derived from, for example, the inner cell mass of blastocysts [Doetchman et al., Dev. Biol. (1988)127:224-227], from inner cell masses [Tokunaga et al., Jpn. J Anim. Reprod. (1989) 35:173-178], from disaggregated morulae [Eistetter, Dev. Gro. Differ. (1989) 31:275-282] or from primordial germ cells [Matsui et al., Cell (1992) 70:841-847]. Transgenic mice can be generated from ES cells which have been treated in accordance with the methods herein by injection of several ES cells into the blastocoele cavity of intact blastocysts [Bradley et al., Nature (1984) 309:225 256]. Alternatively, a clump of ES cells can be sandwiched between two eight-cell embryos [Bradley et al., (1987) in “Teratocarcinomas and Embryonic Stem Cells: A Practical Approach,” Ed. Robertson E. J. (IRL, Oxford, U.K.), pp. 113-151; Nagy et al., Development (1990) 110:815-821]. Both methods result in germ line transmission at high frequency.

In an alternative embodiment, the cells are DNA repair-deficient. The terms “DNA repair-deficient” and “reduced level of at least one DNA repair polypeptide” refer to a quantity and/or activity of a DNA repair polypeptide which is less than, preferably at least 10% less than, more preferably at least 50% less than, yet more preferably at least 90% less than, the quantity and/or activity of the DNA repair polypeptide in a control cell (a corresponding cell type such a wild-type cell which contains the wild-type DNA repair polypeptide), and most preferably is at background level. When a background level or undetectable quantity and/or activity of a DNA repair polypeptide is measured, this can indicate that the DNA repair enzyme is absent. However, a “reduced-level of at least one DNA repair polypeptide” need not, although it can, mean an absolute absence of the DNA repair polypeptide. The method does not require that the DNA repair polypeptide is 100% ablated. The quantity of a DNA repair polypeptide can be determined by, for example, enzyme linked immunosorbent assays. The activity of a DNA repair polypeptide can be determined using methods known in the art for each of the DNA repair enzymes and proteins.

The method is not limited to the type of DNA repair polypeptide whose level is reduced in the cell. Rather, the disclosure expressly contemplates within its scope cells which contain reduced levels of any one or more DNA repair polypeptides that are exemplified by, but not limited to, XPA, XPB, XPC, XPD, XPF, XPG, ERCC1, Cockayne's A, B, DNA glycosylases, Fenl, DNA ligase, Ku 70,86 proteins, DNA Pk, Mrel I complex, XRCC 2,3, ligase IV, XRCCI, polymerase iota, polymerase eta, polymerase zeta, 06-methylguanine-DNA methyltransferases, and DNA photolyase. Preferably, the DNA repair polypeptide whose level is reduced is one that is involved in the repair of the DNA mutation, which is introduced into the cell. For example, cells treated with a molecule that generates double strand breaks preferably contain a reduced level of one or more DNA repair polypeptides (for example, Ku 70,86, Mrel 141 complex, XRCC 2,3, ligase IV, and XRCCI) which are involved in double strand break repair.

Examples of DNA-repair deficient cells contemplated herein include, without limitation, Chinese hamster cells described in Table 2, infra [i.e., UV24 cells, UV5 cells, LTV61 cells, UV41 cells, IRS1 SF cells, XRV15B cells, EM9 cells, and V3 cells]; XP12Be cells (Human) which have a defective XPA gene that results in a defect in damage recognition; MB 19tsA cells (mouse) which have a defective polbeta gene which encodes α-polymerase in the base excision repair pathway; M0595 cells (human), which have a defective DNA Pk gene, which encodes a kinase in the double strand break, repair pathway; UV135 cells (hamster), which have a defective XPG gene that results in a defect in the nucleotide excision repair pathway; UV20 cells (hamster), which have a defective ERCCl gene, which encodes a nuclease cofactor in the nuclease excision repair pathway; GM00671 cells (human), which have a defective XPC gene, which is involved in damage recognition in the nucleotide excision pathway; GM01588 cells (human), which have a defective ATM gene which encodes a damage sensor in end repair and double strand repair pathways; GM02359 cells (human), which have a defective XPV gene, which encodes polymerase eta that functions in lesion bypass; GM00811 cells (human), which have a defective BIM gene, which encodes a helicase; GM13705 cells (human), which have a defective BRCAI gene, which is involved in recombination repair; GM 14170 cells (human), which have a defective BRCA2 gene, which is involved in recombination repair; and AG03141 cells (human), which have a defective WRN gene which encodes a helicase.

The animals from which the target cells are derived are preferably mammalian. In one embodiment, the “mammal” is rodent, primate (including simian and human) ovine, bovine, ruminant, lagomorph, porcine, caprine, equine, canine, feline, ave, etc.

Another embodiment employs the DNA modifying molecules for targeted recombination for the purpose of producing gene knockout organisms and/or of replacement of defective genes with non-defective genes. It is well established that the frequencies of existing protocols for homologous recombination in mammalian cells are quite low. However, using the methods herein, the frequency can be dramatically increased by introduction of a double strand break into the target site. Reagents that are capable of targeting double strand breaks to specific chromosomal sites can be utilized in protocols that include donor DNA and that have as an outcome efficient targeted recombination. This is useful for the construction of transgenic animals and cell lines as described above, and also for the correction of existing gene defects at the site of the defect. For example, in one embodiment, it is contemplated that stem cells (for example, from bone marrow, blood, etc.) are isolated from a patient or host, a defective gene is deleted or repaired, and the cells are transferred back into the patient or host to populate a region in the patient or host for example, the bone marrow with treated stem cells. The present methods can help ensure the efficiency of the mutation and/or recombination phase of the procedure, thereby increasing the likelihood of success. This approach has the advantage of a genetic in situ correction, rather than the current approach of introducing a separate copy of a gene that integrates at a site other than the natural site, and that is often subject to regulation that is different from the native gene. When homologous recombination is desired, standard techniques to select for homologous recombination of a sequence into the matching chromosomal locus can be used (Mansoul et al, Nature (1988) 336:348-352).

In another embodiment, the methods can be used to determine the function of a gene of unknown function. This is of particular interest, given the current concern for characterizing the many new genes described by the Genome Project. For example, if a transgenic animal, which is constructed using the methods herein that result in knockout of a gene of unknown function, were to develop cancer, it could be concluded that the function of the gene was related to the regulation of cellular growth in the tissues in which the cancer originated. Alternatively, if the animal developed muscular disorders it could be concluded that the novel gene played a role in the normal development and function of muscle tissue.

The methods herein result in a frequency of mutation in the target nucleotide sequence of from 0.2% to 7%. However, any frequency of mutation and/or recombination can be useful. Use of cell synchronization coupled with TFO treatment can affect the mutation frequency. For example, unsynchronized cells treated according to the present methods yield a mutation frequency of from about 0.2% to about 3%, but cells treated in S-phase have a higher mutation frequency. In a working embodiment, cells in S-phase treated with a TFO had a mutation frequency of 7%. Any frequency between 0% and 100% of mutation and/or recombination is contemplated to be useful. The frequency of mutation and/or recombination is dependent on the method used to induce the mutation and/or recombination, the cell type used, the specific gene targeted, the DNA modifying molecule used, and the DNA targeting agent used, if any. Additionally, the method used to detect the mutation and/or recombination, due to limitations in the detection method, can not detect all occurrences of mutation and/or recombination. Furthermore, some mutation and/or recombination events can be silent, giving no detectable indication that the events have taken place. The inability to detect silent mutation and/or recombination events gives an artificially low estimate of mutation and/or recombination. Because of these reasons, and others, the methods are not limited to any particular mutation and/or recombination frequency. In one embodiment, the frequency of mutation and/or recombination is between 0.01% and 100%. In another embodiment, the frequency of mutation and/or recombination is between 0.01% and 50%. In yet another embodiment, the frequency of mutation and/or recombination is between 0.1% and 10%. In still yet another embodiment, the frequency of mutation and/or recombination is between 0.1% and 5%.

Without limitation to theory, there appear to be at least three possible explanations for the greater frequency of targeting in S phase. One is that the G₀/G₁ cells are inefficiently electroporated, relative to S phase cells. Analysis of the electroporation efficiencies of the GFP plasmid and the fluorescent oligonucleotides does not appear to support this contention. A second possibility is that the stability of triplexes, once formed, is much greater in S phase cells than G₀/G₁ cells. However, generally triplexes are less stable in cells than in “physiological buffer” in vitro, likely due to cellular enzymes that can disrupt triplexes (46). This suggestion is somewhat counterintuitive since chromatin remodeling is generally associated with gene activation and Hprt gene expression is quite low in quiescent cells (100).

A third possibility is that the levels of TFO binding reflect the accessibility of the target sequence. Triplex formation by 15-20 nucleotide TFOs on nucleosomal sequences is impeded by the requirement of the third strand to occupy the major groove, 8-10 base pairs of which would be exposed with the remainder turned against the histone core complex. In vitro studies indicate that nucleosomal sequences associated with the nucleosomal core are inaccessible to TFOs while those at ends or in internucleosomal linker regions are more available (101, 108-110).

The term “frequency of mutation” as used herein in reference to a population of cells which are treated with a DNA modifying molecule that is capable of introducing a mutation into a target site in the cellular genome, refers to the number of cells in the 32 treated population which contain the mutation at the target site as compared to the total number of cells that are treated with the DNA modifying molecule. For example, with respect to a population of cells that is treated with a DNA targeting agent tethered to psoralen, which is designed to introduce a mutation at a target site in the cells' genome, a frequency of mutation of 5% means that of a total of 100 cells that are treated with DNA targeting agent-psoralen, 5 cells contain a mutation site at the target.

Although the disclosed methods do not require any degree of precision in the mutation and/or recombination of DNA in the cell, it is contemplated that some embodiments will use higher degrees of precision, depending on the desired result. For example, the specific sequence changes required for gene repair, for example, a higher degree of precision is preferred for particular base changes as compared to producing a gene knockout wherein only the disruption of the gene is necessary. With the present methods, achievement of higher levels of precision in mutation and/or homologous recombination techniques is greater than with prior art methods.

Methods for the measurement of DNA mutation and/or recombination in the recipient cell or cells varies depending on the cell type used, the nature of the mutation and/or homologous recombination in the cell and the physiological or morphological effect of the DNA-modifying and/or recombination event. Any method or methods can be used to determine the DNA mutation and/or recombination in the recipient cell or cells. The contemplated methods are well known to those practiced in the art. For example, when the present method is used for modifying the DNA or for the recombination of DNA in a zygote, the recombination event is expected to result in a change or changes in a physiological function or a morphological characteristic in the resulting organism. The expected change or changes can then be assayed or observed. Additionally, DNA samples obtained from the organism and changes in gene sequence can be determined by PCR (see, for example, U.S. Pat. Nos. 4,683,195 and 4,683,202, to Mullis, hereby incorporated by reference).

In the case of cell lines or primary cell cultures, changes in physiology or morphology as the result of the mutation and/or recombination event can be assayed and observed as desired. The assay used is determined by the nature of the physiological change mediated by the mutation and/or recombination event. Additionally, DNA mutation and/or genetic recombination can be assessed using PCR analysis, as detailed above.

Mutation of DNA in cultures of synchronized cells (cells in which the cell cycle has been synchronized) is also disclosed herein. Any method can be used to synchronize the cell cycle. Indeed, a number of different methods are contemplated for synchronization of the cell cycle. For example, the cell cycle can be synchronized at the G2/1 4 boundary by culture with 12-O-tetradecanoyl phorbol-13-acetate (TPA; see e.g., Arita et al., Exp. Cell Res. (1998) 242:381-390), by culture in minimal medium (see e.g., Isakson et al., J. Immunol. Methods (1991)145:137-142), by limited cell attachment time followed by removal of unattached cells (see e.g., Held et al., In Vitro Cell Dev. Biol. (1989) 25:1025-1030), by culture with aphidicolin, or other DNA polymerase inhibitors, to induce an S phase block (see e.g., Matherly et al., J. Biochem. 182:338-345, 1989), by density arrest (see e.g., Takimoto et al., FEBS Lett. (1989) 247:173-176), by double isoleucine block (see e.g., Takimoto et al., FEBS Lett. (1989) 247:173-176), by culture with nocodazole (see e.g., Nusse et al., Cell Tissue Kinet. (1984) 17:13-23) or other microtubule formation inhibitor (e.g. colchicine) or by a combination of one or more of the above mentioned methods (see e.g., Cao et al., Exp. Cell Res. 193:405-410).

Methods for the synchronization of the cell cycle of cultured cells are well known in the art. For example, one method separates rounded, mitotic cells from cultures of attached cells with mild agitation of the culture apparatus. Inhibitors of microtubule assembly such as trypostatin A (Usui et al., Biochem. J. (1998) 333:543-548), phomopsidin (Namikosh et al., J. Antibiot. (Tokyo) (1997) 50:890-892), colchicine and taxol (Sigma Chemical, St. Louis, Mo.) are also used to synchronize cell cycle. Other methods of cell cycle synchronization include thymidine block and DNA synthesis inhibition.

After exposure of the cells to any reagent suitable for cell cycle synchronization, the cells are monitored to determine when the cells are at about the same point in the cell cycle. The cells in the culture progress to the stage in the cell cycle where the cell cycle block takes effect. Once a portion of the cells reach the cell cycle stage where the cell cycle block is effective, the reagent is washed away, usually by repeated gentle rinses (in the case of attachment dependent cells) or by repeated resuspension and centrifugation (in the case of suspension cells). There is no particular percentage of cells necessary to reach the cell cycle stage where the cell cycle blocking agent is effective. However, it is preferred that the portion of the cells at the cell cycle stage where the blocking agent is effective is at least 70%, for example at least 90%. After washing, the cells reinitiate cell cycle progression at about the same rate. At this point the cells are considered to be synchronized. Synchrony is usually lost after several cell cycles as individual cells progress through the cell cycle at slightly differing rates.

Any method can be used to detect cell cycle synchronization. Indeed, a number of methods are contemplated. For example, cell cycle synchronization can be determined by visual observation with light microscopy. Also, synchronization can be detected by staining with DNA intercalating reagents such as propidium iodide or acridine orange. Cell cycle stage can then be determined with fluorescent microscopy of flow cytometry.

Any method can be used to determine the extent of cell cycle synchronization or the phase of the cell cycle population. Several techniques are known to those of ordinary skill in the art to determine the cell cycle of a population of cells. The easiest, but least accurate, method is to look at the cells using light microscopy. The phases of the cell cycle are easily discernable and the extent of synchronization can be determined by counting the percentage of cells at the inhibition point. When most of the cells have been cell cycle inhibited, the agent inducing the inhibition can be washed away thereby allowing the cells to begin cycling in synchrony.

More sophisticated methods for measuring cell cycle synchrony are available and can be more efficient, especially if large numbers of cell populations are to be examined. For example, flow cytometry can be used to determine the cell cycle stage of a sample of cells from a population. The sample is stained with a fluorescent dye that intercalates into the DNA (e.g., propidium iodide and acridine orange). The stained cell sample is then passed through the flow cytometer and a profile is generated that is indicative of the position of the cells in the cell cycle. Flow cytometry has the added advantage of allowing for the easy determination of the percentage of cells in the sample in any particular stage of the cell cycle.

Furthermore, there are many suitable methods to transfect the cells with DNA modifying molecules, such as modified oligonucleotides. Methods for the introduction of DNA modifying molecules into a cell or cells are well known to those of ordinary skill in the art and include, but are not limited to, transduction, microinjection, electroporation, passive adsorption, calcium phosphate-DNA coprecipitation, DEAE-dextran-mediated transfection, polybrene-mediated transfection, liposome fusion, lipofectin, protoplast fusion, retroviral infection, biolistics (particle bombardment) and the like.

One embodiment of the present method includes synchronizing the cell cycle of a plurality of cells, exposing the synchronized cells to at least one DNA modifying molecule, and testing the cells for mutations in their DNA. In another embodiment, the plurality of cells includes cells of more than one cell type. In a culture of multiple cell types, only the cell type of interest need be synchronized. In yet another embodiment, the cells are exposed to the modified oligonucleotide at a specific point in the cell cycle of the synchronized cells. In yet another embodiment, the cell cycle synchronized cells are mammalian. In yet another embodiment, the cell cycle synchronized cells are zygotes.

The disclosed DNA modifying molecules and pharmaceutical compositions thereof can be administered in a number of ways depending on whether local or systemic treatment is desired, and on the area to be treated. Administration can be topically (including ophthalmically, vaginally, rectally, intranasally), orally, by inhalation, or parenterally, for example by intravenous drip, subcutaneous, intraperitoneal or intramuscular injection. The disclosed DNA modifying molecules and pharmaceutical compositions can be administered intravenously, intraperitoneally, intramuscularly, subcutaneously, intracavity, or transdermally.

Preparations for parenteral administration include sterile aqueous or non-aqueous solutions, suspensions, and emulsions which can also contain buffers, diluents and other suitable additives. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives can also be present such as, for example, antimicrobials, anti-oxidants, chelating agents, and inert gases and the like.

Formulations for topical administration can include ointments, lotions, creams, gels, drops, suppositories, sprays, liquids and powders. Conventional pharmaceutical carriers, aqueous, powder or oily bases, thickeners and the like can be necessary or desirable.

Compositions for oral administration can include powders or granules, suspensions or solutions in water or non-aqueous media, capsules, sachets, or tablets. Thickeners, flavorings, diluents, emulsifiers, dispersing aids or binders can be desirable.

In another embodiment, liposomes can be used to deliver the DNA modifying molecules, particularly modified oligonucleotides, to cells. Liposomes can be produced by standard methods such as those reported by Kim et al., Biochim. Biophys. Acta (1983) 728:339-348; Liu et al., Biochim. Biophys. Acta (1992) 1104:95-101; and Lee et al., Biochim. Biophys. Acta. (1992) 1103:185-197; Wang et al., Biochem. (1989) 28:9508-9514). Such methods have been used to deliver nucleic acid molecules to the nucleus and cytoplasm of cells of the MOLT-3 leukemia cell line (Thierry and Dritschilo, Nucl. Acids Res., (1992) 20:5691-5698). In another embodiment, the DNA modifying molecules can be incorporated within microparticles, or bound to the outside of the microparticles, either ionically or covalently.

In another embodiment, cationic liposomes can be used to deliver DNA modifying molecules, particularly modified oligonucleotides and other negatively charged molecules. Cationic liposomes or microcapsules are microparticles that are particularly useful for delivering negatively charged compounds, which can bind ionically to the positively charged outer surface of these liposomes. Various cationic liposomes have previously been shown to be very effective at delivering nucleic acids or nucleic acid-protein complexes to cells both in vitro and in vivo, as reported by Felgner et al., Proc. Natl. Acad. Sci. USA (1987) 84:7413-7417; Felgner, Advanced Drug Delivery Reviews (1990) 5:163-187; Clarenc et al., Anti-Cancer Drug Design (1993) 8:81-94. Cationic liposomes or microcapsules can be prepared using mixtures including one or more lipids containing a cationic side group in a sufficient quantity such that the liposomes or microcapsules formed from the mixture possess a net positive charge, which will ionically bind negatively charged compounds. Examples of positively charged lipids that can be used to produce cationic liposomes include the aminolipid dioleoyl phosphatidyl ethanolamine (PE), which possesses a positively charged primary amino head group; phosphatidylcholine (PC), which possess positively charged head groups that are not primary amines; and N[1-(2,3-dioleyloxy)propyl]-N,N,N-triethylammonium (“DOTMA,” see Felgner et al., Proc. Natl. Acad. Sci. USA (1987) 84:7413-7417; Felgner et al., Nature (1989) 337:387-388; Felgner, Advanced Drug Delivery Reviews (1990)5:163-187).

The dosage ranges for the administration of the DNA modifying molecules are those large enough to produce the desired effect in which delivery occurs. The dosage should not be so large as to cause adverse side effects, such as unwanted cross-reactions, anaphylactic reactions, and the like. Generally, the dosage will vary with the age, condition, sex and extent of the disease in the patient and can be determined by one of skill in the art. The dosage can be adjusted by the individual physician in the event of any counter indications. In one embodiment, the dosage can vary from about 1 mg/kg to 30 mg/kg in one or more dose administrations daily, for one or several days. The dose, schedule of doses and route of administration can be varied, whether oral, nasal, vaginal, rectal, extraocular, intramuscular, intracutaneous, subcutaneous, or intravenous, to avoid adverse reaction yet still achieve delivery.

### III. EXAMPLES

The foregoing disclosure is further explained by the following non-limiting examples. Efforts have been made to ensure accuracy with respect to numbers (e.g., amounts, temperature, etc.) but some errors and deviations should be accounted for. Unless indicated otherwise, parts are parts by weight, temperature is in ° C. or is at ambient temperature and pressure is at or near atmospheric.

Methods

Synthesis of 2′-AE-5-methyluridine CPG (Scheme 1)

5′-O-(4,4′*dimethoxytrityl)-5-methyluridine-2′-O-(2-aminoethyl)-3′-O-Succinate (44) 2: 5′-O-(4,4′-dimethoxytrityl)-5-methyluridine-2′-O-(2-aminoethoxy) (1, 0.45 g, 0.643 mmol), which was prepared by prior art techniques (39, 40), was dissolved in anhydrous dichloromethane (5 ml), and 0.128 g (1.28 mmol) succinic anhydride was added to the solution. Subsequently, 78.1 mg (0.64 mmol) of 4-(dimethylamino)pyridine (DMAP) was added, and the mixture was stirred for 90 min. After completion of the reaction (as indicated by TLC), the solvent was evaporated in vacuo. The residual yellow oil was dissolved in dichloromethane and washed twice with 10% NaHCO₃(aq). The extracted organic phase was dried over Na₂SO₄, and evaporated in vacuo to yield a white solid 2 (0.43 g, 85%). MS (HR-FAB) m/z 821.726 (M+Na)⁺.

5′-O-(4,4′-dimethoxytrityl)-5-methyluridine-2′-O-methyl-3′-O-succinimido-N⁶-hexanamido-N³-propyl-controlled pore glass (CPG) 3: The 3′-succinate block (2) (128 mg, 0.16 mmol) was coevaporated with anhydrous pyridine and then dissolved in anhydrous DMF (2 ml). Subsequently, 85 μL (0.5 mmol) of N,N-diisopropylethylamine (DIEA) was added. A solution of 76 mg (0.2 mmol) HATU in DMF was added to the mixture while stirring. Stirring was continued for about 1 min to allow preactivation before the mixture was added to 1.25 g of acid treated LCAA-CPG (initial loading: 90 μmol/g), and the suspension was shaken for 16 h. Subsequently, the resin was washed with DMF, DCM, and CH₃CN. The unreacted amino groups of the resin were capped by shaking the resin with 0.36 mL (3 mmol) of ethyl trifluoroacetate and 0.42 mL (3 mmol) of TEA in 6 mL of MeOH. Finally, the resin was washed with MeOH, CH₃CN, and DCM and dried in vacuo. The loading on the CPG was determined by DMT release assay (final loading=30 μmol/g).

Synthesis of Modified oligonucleotides: The 5′-O-(4,4′-dimethoxytrityl)-5-methyluridine-2′-O-methyl-3′-O-(β-cyanoethyl-N,N-diisopropyl)phosphoramidite, the N⁴-formamidine-5′-O-(4,4′-dimethoxytrityl)-5-methylcytidine-2′-O-methyl-3′O-(β-cyanoethyl-N,N-diisopropyl)phosphoramidite, the 5′-O-(4,4′-dimethoxytrityl)-5-methyluridine-2′-O-methyl-3′-O-succinimido-N⁶-hexanamido-N³-propyl-controlled pore glass (CPG) support and the 6-[4′-(hydroxymethyl)-4,5′,8-trimethylpsoralen]hexyl-1-0-(β-cyanoethyl-N,N-diisopropyl)phosphoramidite were purchased from Chemgenes, Ashland, Mass. For the synthesis of 5′-O-(4,4′-dimethoxytrityl)-5-methyluridine-2′-O-(2-aminoethoxy)-3′-O-(P-cyanoethyl-N,N-diisopropyl)phosphoramidite and N⁴-(N-methylpyrrolidineamidine)-5′-O-(4,4′-dimethoxytrityl)-5-methylcytidine-2′-O-(2-aminoethoxy)-3′-O-(β-cyanoethyl-N,N-diisopropyl)phosphoramidite previously reported procedures were followed (39; 40; 45). The oligonucleotides were synthesized on CPG supports (500 Å) using an Expedite 8909 synthesizer using previously described procedures (43).

The conditions for Scheme 1: (i) Nucleoside 1, succinic anhydride (2 equiv.), DMAP [4-(dimethylamino)pyridine, 1 equiv.], CH₂Cl₂, RT, 1 h; (ii)LCAA-CPG (long-chain alkylamine controlled pore glass, initial loading: 90 mol/g), Succinate 2 (1.5 equiv.), HATU [O-(7-azabenzotriazol-1 yl)-1,1,3,3-tetramethyluronium hexafluorophosphate, 2 equiv.], DIEA (N,N-diisopropylethylamine, 5 equiv), DMF, r.t. 16 h; final loading as determined by DMT-assay: 3, 30 mol/g.

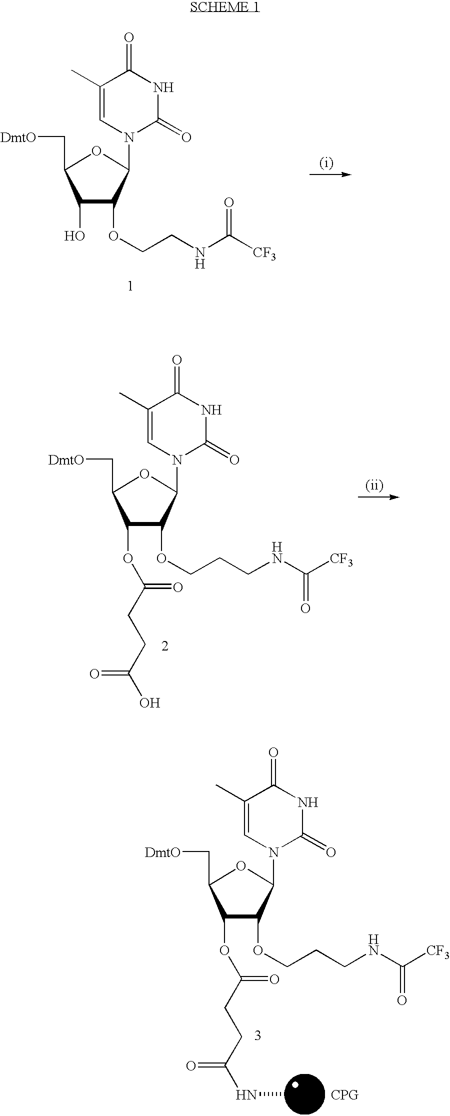

Deprotection and Purification of Modified oligonucleotides: The controlled pore glass support with a Pso-modified oligonucleotide was placed in a vial closed with a porous filter cap for gas phase deprotection (43). The vial was inserted in an enclosed steel pressure chamber with a valve and evacuated by house vacuum. The valve was then connected to the gas cylinder, keeping the steel chamber under reduced pressure. The chamber was incubated with anhydrous methylamine gas (Aldrich) at room temperature for 45 min, followed by the release of the methylamine gas. The modified oligonucleotide was then taken up in distilled water. Analytical and semi-preparative anion exchange (IE)-HPLC was carried out using a Dionex DNAPac PA-100 column (4.0×250 mm and 9.0×250 mm respectively) on a Shimadzu HPLC system (LC-10ADvp) with a dual wavelength detector (SPD-10AVvp) and an autoinjector (SIL-10ADvp). The column was eluted using linear gradients of sodium chloride (0-1.0 M) in 0.1 M Tris buffer (pH 7.0) at a flow rate of 1.0 ml/min and monitored at wavelengths 254 and 315 nm (λₘₐₓ for psoralen). The purified oligos were characterized by capillary zone electrophoresis and matrix-assisted laser desorption-time of flight.

Thermal Denaturation Experiments: The modified oligonucleotide and the constituent strands of the target duplexes (5′ TCAGAAGAAAAAAGAGAAA; 5′ TTTCTCTTTTTTCTTCTGA) were taken in buffers A-D [50 mM Tris, 100 mM NaCl, and 2 mM MgCl₂ (pH 6.0, 6.5, 7.0 and 7.5 respectively)]. These solutions were heated at 80° C. for 3 min, and allowed to come to RT in 30 min. The modified oligonucleotide:duplex target solutions were incubated at 4° C. overnight. The thermal denaturation experiments were carried out in buffers A-D using a Cary 3E UV-visible spectrophotometer fitted with a thermostat sample holder and temperature controller. Triplexes were heated from 10 to 85° C. at a rate of 0.4° C./min, and the absorbance at 260 m was recorded as a function of the temperature. The data was processed using SigmaPlot™ 5.0 software to determine the 1^{st} derivative of the melting curves and the Tm value was obtained. Each analysis was done two times and the error was no more than 0.5° C.

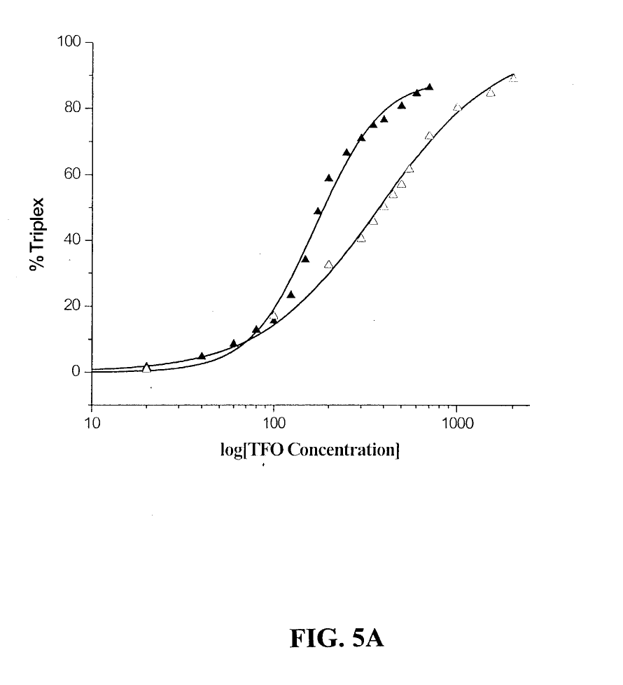

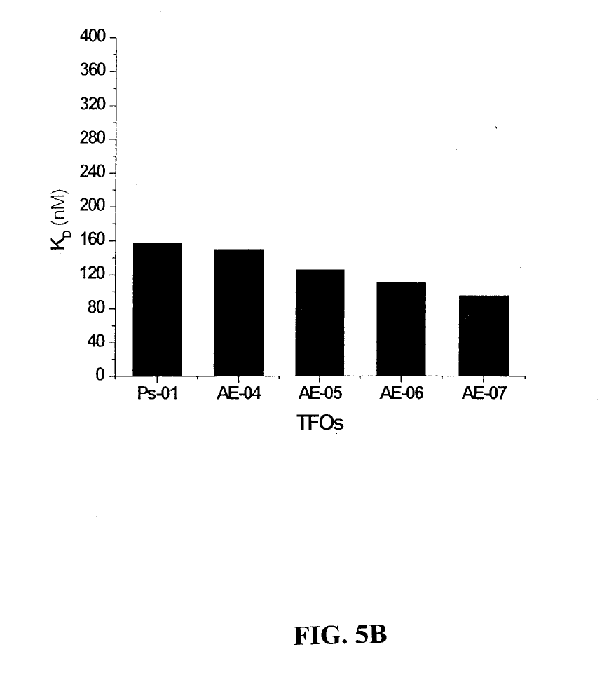

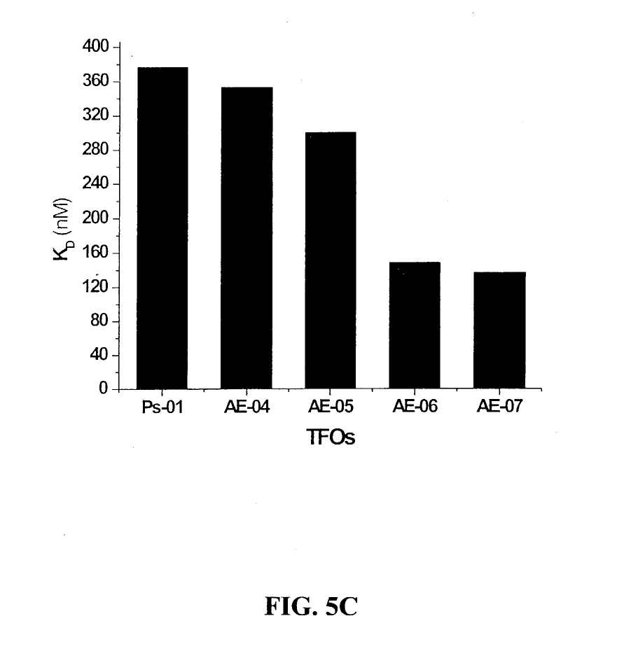

Kd Determination: A snapback duplex oligonucleotide containing the Chinese Hamster Intron4/exon5 HPRT target sequence (5′AGTAGAAGAAAAAAGAGAAATGATTTTCATTTCTCTTTTTTCTTCTACT) was synthesized and labeled by incubation with T4 DNA kinase and ³²P ATP. The radioactive oligonucleotide at a final concentration of 100 pM was incubated with various concentrations of the modified oligonucleotides in buffer consisting of 20 mM Hepes, pH 7.2, and 10 or 1 mM MgCl₂ as indicated, at RT for 24 hrs. Samples were then loaded on a 15% acrylamide gel in Hepes buffer, pH 7.2, containing the appropriate level of MgCl₂ and electrophoresed for 16 hrs. The relative intensity of the duplex and triplex bands was determined by Phosphorimager analysis. The K_{D} values were determined by Hill's equation [y=ax^{b}/(c^{b}+x^{b})], where y=% triplex formation, x=modified oligonucleotide concentration, a=maximum % value of triplex formation, b=Hills coefficient (see legend of FIG. 5b,c), c=approximate value of K_{D}. The assumptions on the basis of which the Hill's equation was used are (1) modified oligonucleotides do not interact with themselves, and (2) the concentration of the duplex target is too small to influence the equilibrium of triplex formation. The data were processed with SigmaPlot™ 5.0 software.

In vivo stability: Details of this procedure have been described previously (46). The method measures the stability of a preformed triplex following introduction of the complex into cells. The psupF12 shuttle vector plasmid was engineered to contain the CHO HPRT triplex binding site in the pre-tRNA portion of a variant supF mutation reporter gene, immediately adjacent to the mature gene sequence (see (43) for the schematic of the supF12 gene). The first two bases of the mature gene sequence were 5′ TA, which is a sequence appropriate for psoralen crosslinking. A psoralen linked modified oligonucleotide, by forming a triplex with the target sequence, positions the psoralen at the TA step such that a crosslink is introduced upon photoactivation. Replication and/or repair of the crosslink in mammalian cells will introduce mutations at the site and these can be quantitated in a microbiological screen of the supF gene in the progeny plasmids, following recovery from the mammalian cells. A crosslink is required for mutagenesis, photoactivation is required for crosslinking, and the positioning of the psoralen is dependent on an intact triplex. Thus the mutation frequency is a reflection of the triplexes present in the plasmid population at the time of photoactivation. Triplexes were formed by incubation of a modified oligonucleotide and the psupF12 plasmid (20 mM Hepes, pH 7.2, 10 mM MgCl₂, 2 μM modified oligonucleotide, 0.6 pmol of plasmid), unbound modified oligonucleotide removed, and the plasmid-modified oligonucleotide complexes electroporated into Cos-1 cells. At various times after transfection the cells were exposed to long wave ultraviolet light (365 nm, 3 min, 1.8 J/cm², in a Rayonet chamber). The cells were incubated for an additional 48 hr, during which time the psoralen cross-links were repaired, some with mutational consequences, and the plasmid replicated. Progeny plasmids were then harvested, treated with DpnI to remove nonreplicated input plasmids (47), and introduced into the Escherichia coli indicator strain MBM 7070 (48). The bacteria were spread on plates containing isopropyl-1-thio-β-D-galactopyranoside and 5-bromo-4-chloro-3-indolyl-β-D-galactopyranoside, and the frequency of white or light blue colonies, which contained plasmids with mutations in the supF gene, was determined.

HPRT knockout assay: Chinese Hamster Ovary (CHO) cells were grown in Dulbecco's modified Eagle medium (DMEM) supplemented with penicillin, streptomycin, glutamine, and 10% fetal calf serum. Cells were cultured in HAT medium (10⁻⁴ M hypoxanthine, 5×10⁻6 M aminopterin, 10⁻⁵ M thymidine) for 1 week to remove pre-existing HPRT (hypoxanthine phosphoribosyl transferase) deficient cells. They were treated as described previously (49), a procedure that enhances knockout activity and will be described in a future publication. The modified oligonucleotides were introduced by electroporation (BIORAD, 130 volts, 960 microfarads) with 3×10⁶ cells suspended in 200 μl with each modified oligonucleotide at 5 μM. They were incubated at room temperature for 3 hr, and exposed in a Rayonet chamber to UVA light for 3 min at 1.8 J/cm². The cells were plated in complete medium for 8-10 days with 2-3 passages, and then placed in selective medium depleted of hypoxanthine and containing 20 μM thioguanine (200,000 cells/i 00-mm dish). Cells were also plated in selective medium without thioguanine to determine plating efficiency. After 10 days resistant colonies were counted and picked for expansion and DNA analysis.

A similar protocol was observed when the frequency of cells with mutations in APRT (adenosine phosphoribosyltransferase) was determined. CHO-AT3 (hemizygous for APRT) cultures were cleared of APRT deficient cells by growth in 10 μM azaserine, 20 μM adenine. Selections for APRT deficient colonies were done in medium containing 50 μg/ml aza-adenine.

Synchronization protocol: Chinese Hamster Ovary (CHO) AA8 cells (ATCC) were prepared as described above for the HPRT knockout assay. The cells were synchronized in G₀/G₁ by a variation of the method described by Sawai et al. (96). Briefly, cells were plated at subconfluent levels and the next day the medium changed to DMEM with 2% FBS and 2% DMSO. After 48 h the cells were washed (˜85% G₀/G₁ cells by FACS analysis) and either electroporated or fed with complete medium (for G₁ phase experiments), or incubated with complete medium containing 100 μM mimosine for 16 hrs to block them in early S phase (˜90% early S cells). See Orren et al. for complete details on this procedure (49). After 16 hrs the cells were released from the mimosine block by feeding with DMEM/10% FBS.

TFO electroporation, Psoralen treatment, and Hprt mutation assay: Cells were suspended at 10⁷/mL and mixed with TFO at 5 μM. The cells were electroporated (BioRad), followed by incubation at room temperature for 3 hrs, and a 3 min exposure in a Rayonet chamber to UVA light at 1.8 J/cm². The electroporation conditions were chosen to minimize cell toxicity (trypan blue staining showed ˜95% viable cells after UVA treatment). Cells treated with free psoralen were incubated with 5 μM psoralen for 30 min, followed by UVA treatment. The cells were passaged and then exposed to thioguanine (TG) selection (43).

Restriction resistance of non-selected clones: Following TFO electroporation, UVA treatment and culture for 3-5 days to permit mutagenesis, 100 cells were plated in 60 mm dishes in standard growth medium. Individual colonies were expanded, the DNA extracted, followed by amplification of the 14E5 target region and digestion of the PCR products with XbaI.

Crosslink analysis: After UVA exposure of cells treated with the pso-TFO the DNA was extracted and digested with EcoRI. Then 10 μg, in 10 μL, were mixed with 45 μl of 98% formamide and heated at 77° C. for 15 min. The samples were electrophoresed in an agarose gel in neutral buffer and transferred to a nylon membrane. The membrane was hybridized with a 2 kb EcoRI fragment containing the 14E5 target region and adjacent sequence. The probe was labeled by random priming and incubated overnight with a blank membrane in hybridization buffer to remove radioactive species that bound nonspecifically. This step greatly reduced non-specific binding to the nylon. In other experiments, following TFO/UVA treatment, genomic DNA was exhaustively digested with EcoRI and XbaI, blotted and hybridized. To control for triplex formation resulting from the interaction of unbound TFO and the target during DNA purification (31) AE-07 and cells were mixed and then genomic DNA was isolated and digested. No XbaI resistant band was observed.

Results

The target sequence and location in the Chinese Hamster HPRT gene are shown in FIG. 1a. Also indicated are the sequences and substitution patterns of the oligonucleotides described in this report. Each modified oligonucleotide contained 5-methylcytosine, and all sugar residues were either 2′-OMe or 2′-AE (FIG. 1b). All modified oligonucleotides were linked to psoralen as described previously (43).

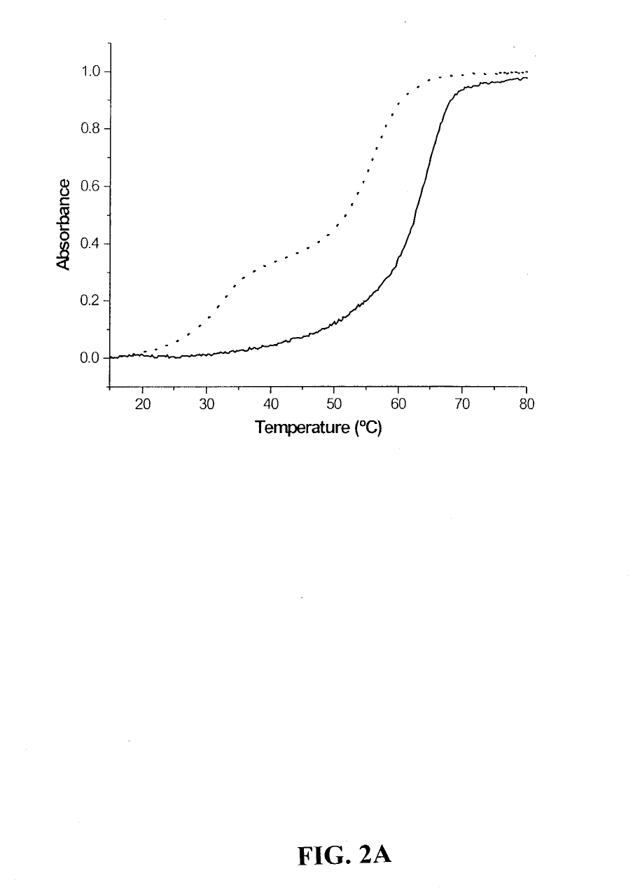

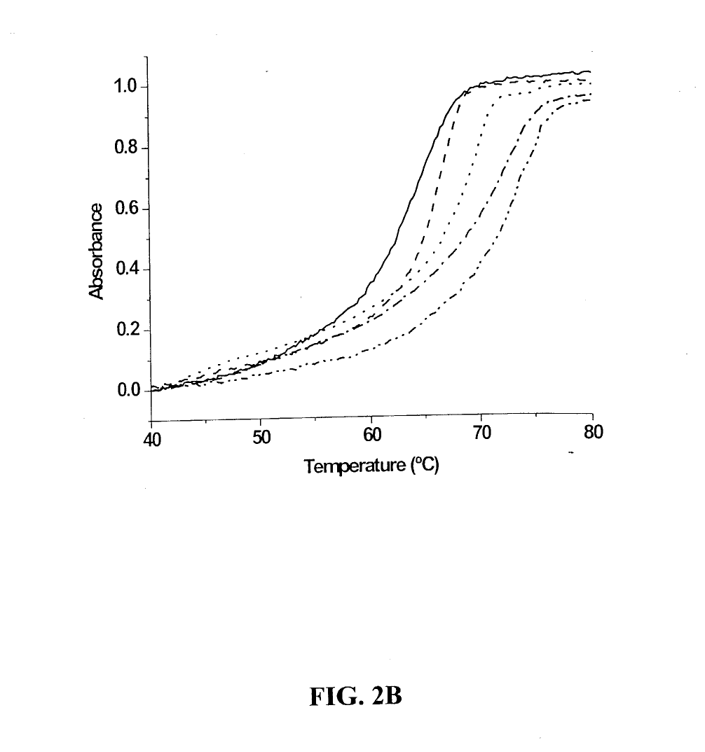

Thermal stability of triplexes formed by 2′-AE Modified oligonucleotide: Triplexes were formed by incubation of individual modified oligonucleotides with a 19 mer duplex oligonucleotide (Methods) containing the 14/E5 CHO HPRT target sequence in buffers ranging from pH 6.0 to 7.5. Gel shift analysis demonstrated the formation of triplexes by each modified oligonucleotide. The thermal stability of the triplexes in each buffer (Materials and Methods) was measured. The analysis, in pH 7.0 buffer, of the triplex formed by the duplex and a third strand containing deoxyribose sugars showed two transitions with the triplex Tm (31.3° C.) below the duplex Tm (55.1° C.) (FIG. 2a). In contrast, the triplex formed by the oligonucleotide containing all 2′-OMe sugars (PS-01) showed a single transition with a Tm (63.8° C.) higher than the duplex Tm. This result demonstrated that the presence of the 2′-OMe ribose in the third strand had a profound effect on triplex stability.

The profiles, at pH 7.0, of the triplexes formed by the modified oligonucleotides containing 2′-AE residues are shown in FIG. 2b. Increased 2′-AE content yielded progressive increase in Tm (˜2.0° C./2′-AE residue) (39), and there was a single transition with each triplex. For the sake of economy the modified oligonucleotides examined in the thermal melting study were those used in the biological experiments. There was concern that possible photoactivation of the psoralen in the spectrophotometer would compromise interpretation of the results. However control experiments showed that modified oligonucleotides without psoralen displayed the same melting profiles, while deliberate photoactivation of the psoralen after triplex formation resulted in profiles with transitions 15° C. above those shown here. Consequently, it was concluded that psoralen was not photoactivated in the cuvette and the presence of the psoralen had no effect on the Tm values.

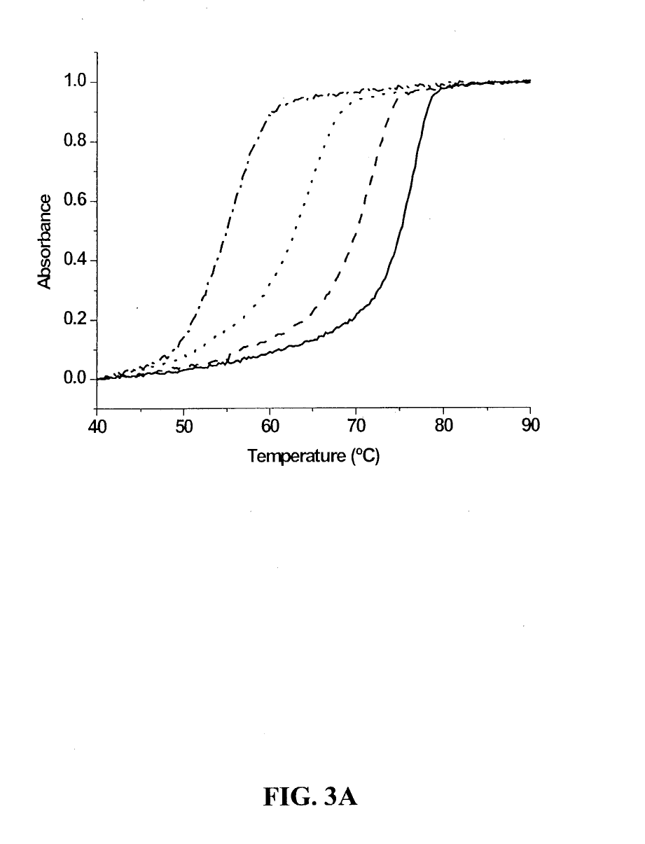

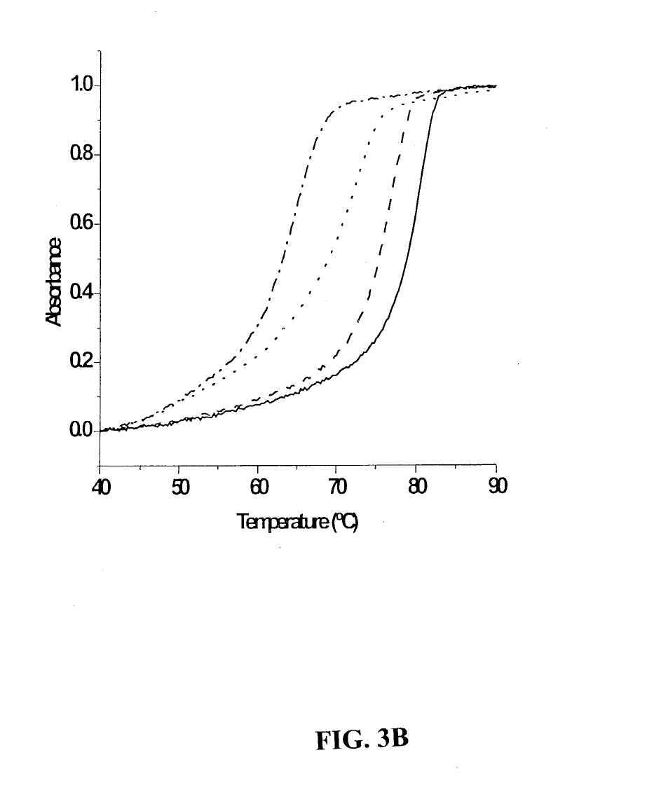

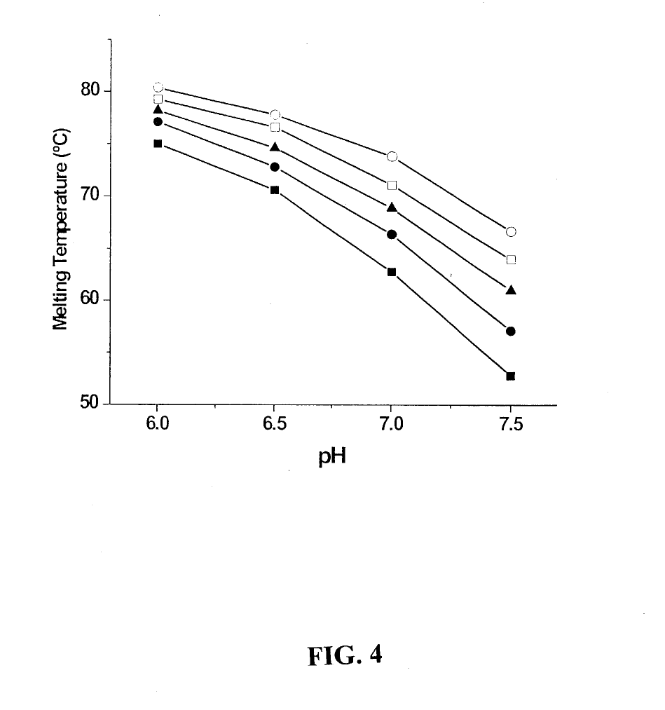

A hallmark of pyrimidine motif triplexes is their sensitivity to increase in pH. Analysis of the triplexes formed by the PS-01 modified oligonucleotide as a function of pH (from 6.0-7.5) showed the predictable decline in Tm as the pH was increased (FIG. 3a). (In control experiments the Tm of the duplex was indifferent to pH.) In all curves there was a single species and only at the highest pH was this single transition coincident with that of the duplex shown in FIG. 2a. The same analysis with the triplex formed by the modified oligonucleotides with AE substitutions also showed declines in Tm as the pH rose, although even at pH 7.5 all modified oligonucleotides had Tm values greater than the duplex (FIG. 3b). Furthermore, as the 2′-AE content increased, the Tm differential (low to high pH) narrowed. The plot of Tm vs. pH for triplexes formed by each modified oligonucleotide showed that the relative decline in Tm for the triplexes formed by the 2′-AE modified oligonucleotides was not as great as that of the PS-01 triplex (FIG. 4). When the pH was raised from 6.0 to 7.5 the decline in Tm was 15.2° C. for AE-06 and 14.2° C. for AE-07, while for PS-01 it was 20.7° C. Thus the presence of the 2′-AE residues mitigated the destabilizing effects of pH in the physiological range.

Aminoethoxy residues enhance modified oligonucleotide affinity: The K_{D} of the modified oligonucleotides in gel shift assays was measured (see Methods section above). The determinations were made in buffers at pH 7.2 containing either 10 or 1 mM MgCl₂. The latter condition was of particular interest because the level of free Mg⁺⁺ in cells is believed to be much lower than 10 mM. The binding isotherms for PS-01 and AE-06 in 1 mM MgCl₂ are shown in FIG. 5a. AE-06 had a lower K_{D} (148 nM) than PS-01 (376 nM). The values of K_{D} for each of the modified oligonucleotides at 10 and 1 mM MgCl₂ are shown in FIGS. 5b and c. The presence of 2′-AE residues resulted in a decrease in K_{D} (relative to PS-01) at both MgCl₂ concentrations, although this was more pronounced in 1 mM MgCl₂. Interestingly, in the lower concentration of MgCl₂, the K_{D} values of AE-06 and AE-07 were lower than would have been expected from the simple proportionality predicted by the results with the modified oligonucleotides with 0, 1, or 2, 2′-AE residues. The assay was also performed in 1 mM MgCl₂ with M AE-06, which had the same three adjacent 2′-AE residues as in AE-06, but a single change in sequence (a C instead of T in the middle of the oligonucleotide). The affinity of this modified oligonucleotide was very poor, and half maximal binding was not observed at 4 μM, the highest concentration used in the assay.

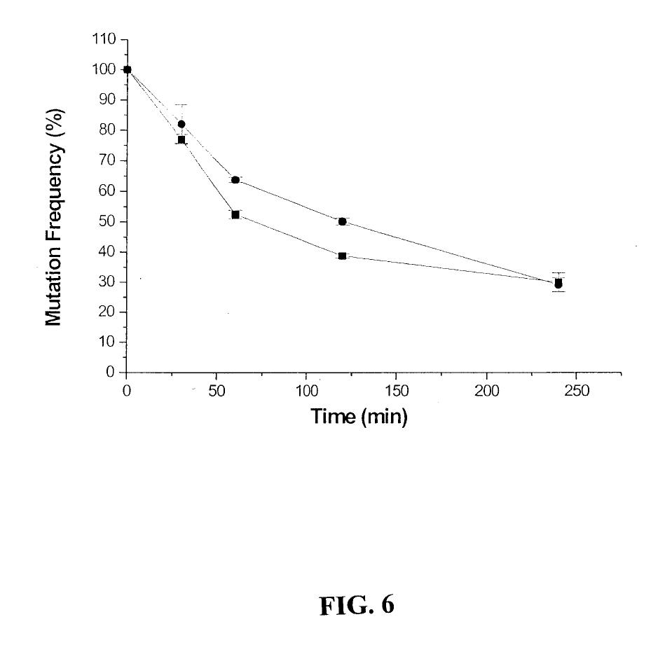

Triplexes formed by modified oligonucleotide AE-06 are more stable in vivo: A method for measuring the stability of triplexes, preformed in vitro, in the cellular compartment that supports replication and mutagenesis has been developed (46). The assay is based on a shuttle vector plasmid carrying a variant supF gene that serves as a mutation marker, and contains the CHO HPRT triplex targeted embedded in the gene. Preformed triplexes on the plasmid were electroporated into mammalian cells and, as a function of time after introduction, the cells were exposed to UVA light to trigger psoralen crosslinking of the plasmids containing triplexes. Plasmids that lost the triplex would not be crosslinked and would not incur mutations. The stability of triplexes formed by PS-01 and AE-06 were compared. As shown in FIG. 6, the triplex formed by AE-06 (t_{1/2}=120 min) was twice as stable in the cells as the PS-01 triplex (t ½=59 min). These data indicated that the triplex formed by the modified oligonucleotide with three AE residues was more resistant to the cellular environment than the PS-01 triplex. Surprisingly, the AE-06 triplex was more stable in vivo than those formed by the modified oligonucleotides with more extensive 2′-AE substitution (for example AE-02, with 6 residues) (43).

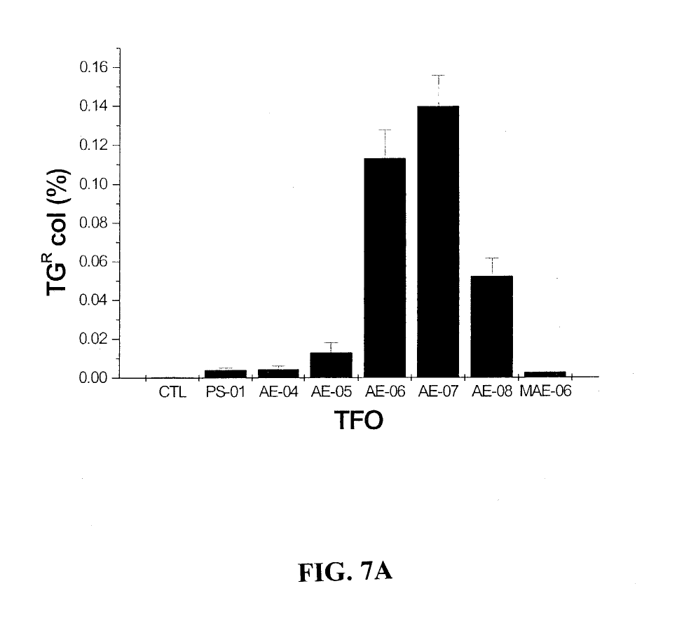

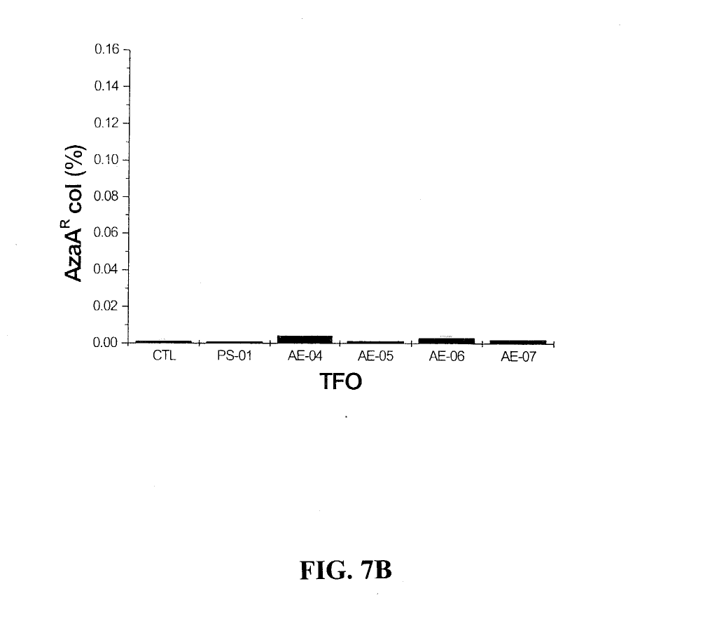

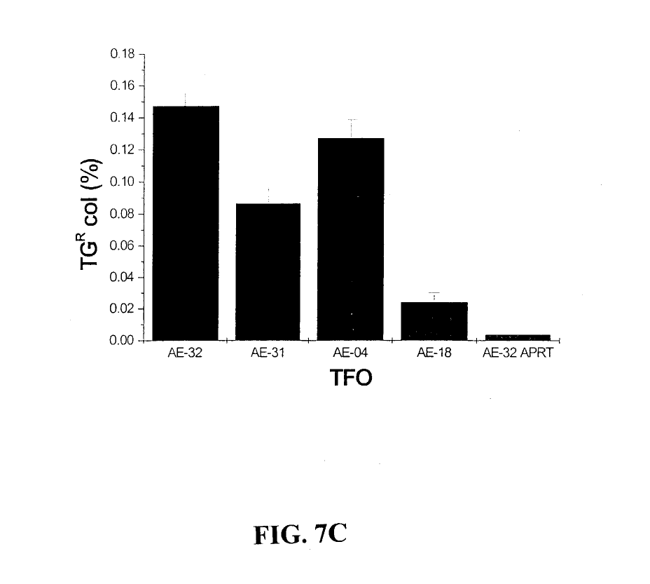

Activity of 2′-AE modified oligonucleotides in the HPRT knockout assay: The pso-modified oligonucleotides were introduced into CHO cells by electroporation (Experimental Methods). After 3 hrs the cells were exposed to UVA, cultured for 8-10 days, and then plated in medium containing 6-thioguanine. Resistant colonies were counted and the results are shown in FIG. 7a. Cells treated with the modified oligonucleotide containing only 2′-OMe sugars, or the modified oligonucleotide with a single 2′-AE substitution showed few 6-TG resistant colonies relative to mock electroporated control cultures. A few fold more colonies were recovered when the modified oligonucleotide with 2 substitutions was tested. In contrast there was a substantial increase in activity with AE-06 and AE-07, with the frequency of TG resistant colonies slightly more than 0.1%, and 0.14% respectively, approximately 300-400 fold over the background. It should be noted that the background mutation frequency reflects the occurrence of inactivating spontaneous mutations across the entire coding region of the HPRT gene, quite different from the localization of events targeted by the modified oligonucleotide. In this example, UVA treatment was provided as a signal, indicating that psoralen activation induced mutagenesis. A single, central, base change in the modified oligonucleotide sequence abolished activity (M AE-06).

Analysis of modified oligonucleotides with a variable patch of three 2′AE residues revealed that 5′ (AE32), middle (AE31), or 3′ (AE04) have similar activity (FIG. 7c). However, the dispersal of the three 2′-AE residues (one 5′, middle, 3′-AE18) consistently showed lower activity. The activity of the AE32 was also tested against another gene lacking the specific target sequence (AE32-APRT). There was no appreciable activity against this target.

To test for possible activity of the modified oligonucleotides against unintended targets the experiment was repeated with the cells exposed to selection for mutations in the APRT gene. However, APRT deficient cells at frequencies little different from background levels were recovered (FIG. 7b). These results suggest that the modified oligonucleotide targeting was specific to the intended HPRT target, at least as monitored at the level of another gene. This was further supported by analysis of the mutations in TG^{R} clones which showed the same events reported previously-principally small deletions, all in the target region (not shown, but see (43)).

2′-AE-Cytidine vs. 2′-AE-Thymidine: AE-06 contains three 2′-AE substituted nucleotides, two thymidines and one cytidine. Cytidines can be at least partially protonated when in pyrimidine motif triplexes even under physiological conditions (18). Therefore, measured the activity of a modified oligonucleotide containing three adjacent AE-thymidines in which the only positive charge in the 2′-AE patch would be contributed by the sugar modification. For comparison, AE-08 was prepared, this oligonucleotide contained three 2′-AE-thymidines at the 3′ terminus of the oligonucleotide. This required synthesis of the 2′-AE-thymidine-CPG support as described above in the methods section. The K_{D} of this modified oligonucleotide (160 nM, 1 mM Mg⁺⁺) and the thermal stability of the AE-08 triplex (70.3° C., pH 7.0) were similar to the data with AE-06. However this modified oligonucleotide quite reproducibly showed about 50% the activity of AE-06 in the HPRT assay (FIG. 7a), indicating that the 2′-AE cytosine offered a measurable enhancement to the bioactivity of the modified oligonucleotide. Thus, the bioassay could distinguish between the two modified oligonucleotides while biochemical measurements did not. This can be the result of the environment in which triplex formation occurs (ionic composition, local pH, etc) or a reflection of enzymatic functions that stabilize or destabilize triplexes, or those that convert the targeted crosslink into a mutation.

The CHO 14/E5 HPRT target and pso-TFO: The triplex target sequence is in the fourth intron, next to Exon 5 of the CHO Hprt gene (FIG. 1a). The sequence consists of a 17 base polypurine:polypyrimidine sequence ending in a 5′ TA step which is appropriate for T-T interstrand crosslinking by psoralen (111). The A is the first base of the AG splice acceptor sequence, and point mutations at this site have been reported (121). The intron/exon junction also contains a recognition sequence for the restriction enzyme XbaI (TCTAGA). The TFO used in these studies, AE-07, was linked to psoralen and contained a patch of four 2′-AE substituted nucleotides, while the remainder of the molecule contained 2′-OMe sugars (FIGS. 1a, b). The synthesis and characterization of TFOs containing 2′-AE substitutions have been described (43, 112). The 2′-AE residues are protonated at physiological pH. They reduce the charge repulsion between the third strand and the duplex target, thus lowering the requirement for Mg⁺⁺ (39, 19). They also establish a stabilizing interaction with phosphates in the purine strand of the duplex target (42). Psoralen linked TFOs containing the appropriate amount and distribution of this modification are active in the Hprt assay (112).

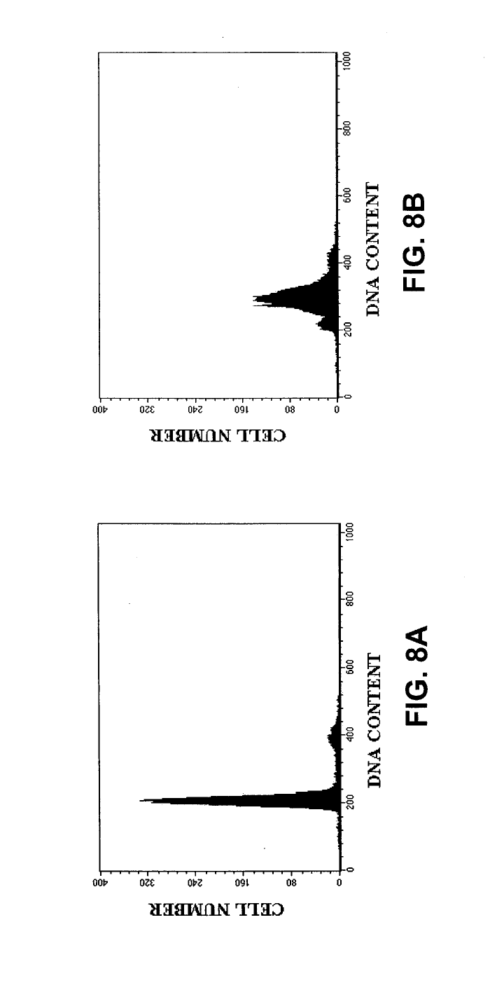

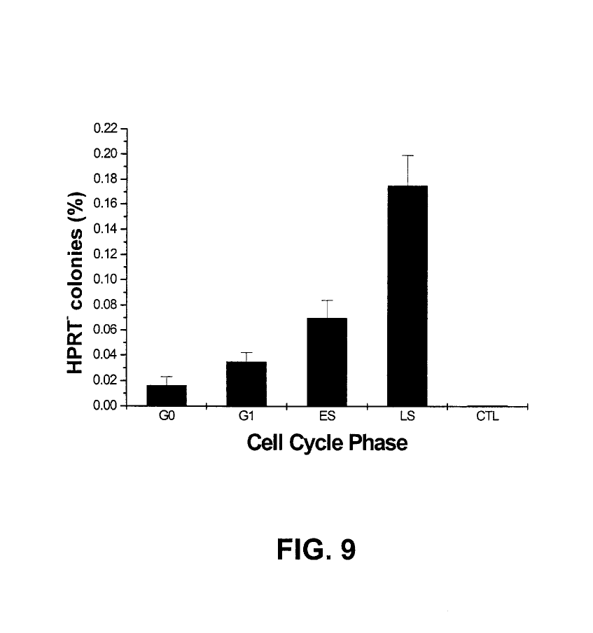

Pso-TFO Activity during the cell cycle: AE-07 was introduced by electroporation into quiescent CHO cells, and into cells at different times after release from quiescence (Methods, see FIG. 8 for FACS profiles of quiescent and mid S phase cells). The psoralen was photoactivated and the frequency of cells with inactivating mutations in the Hprt gene determined via conventional thioguanine selection. The mutation frequency was relatively low in the cells in G₀/G₁, increased in cells traversing G₁, and appeared maximal in S phase cells. To more precisely define the time of peak activity in S phase the cells were released from G₀/G₁ block and then blocked again in early S phase by treatment with complete medium containing mimosine (49). The cells were released from this block and, as before, treated at various times with AE-07/UVA, followed by determination of TG resistant colonies. The greatest activity was seen 4-6 hrs after release, during which time the cells were in mid to late S phase. Results from selected time points are shown in FIG. 9. The mutation frequency of cells treated in mid S phase was 0.15-0.22%, while the level in quiescent cells was 0.02-0.03%.

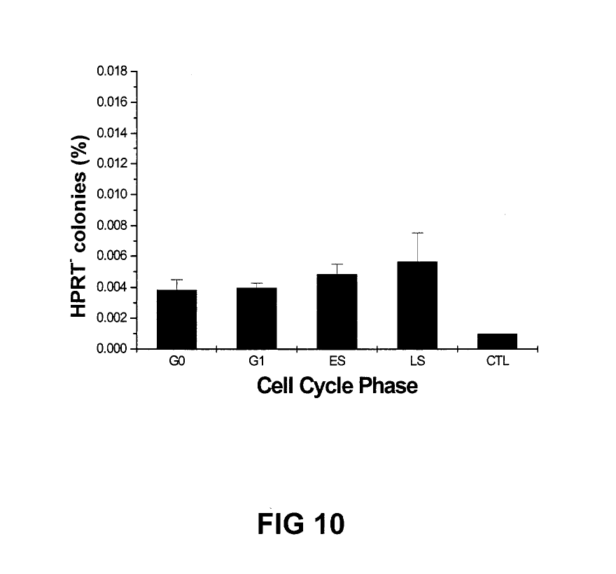

Psoralen mutagenesis during the cell cycle: The results indicated that appearance of mutant cells occurred only after photoactivation (16). Thus the actual mutagen was the psoralen crosslink, targeted to the desired sequence by the associated TFO. It was then determined if Hprt mutagenesis by free psoralen would show cell cycle variability. Cells were treated at different phases of the cell cycle with psoralen as described in the above Methods section and processed as before. Thioguanine resistant colonies were recovered at frequencies a few fold over the background (0.004-0.005% vs 0.001%), consistent with similar experiments with psoralen by other groups (113, 114). However, in contrast to the previous experiment, the mutation frequency was essentially unchanged across the cell cycle (FIG. 10).

Electroporation controls and specificity: Several control experiments were performed. Cells at different times of the cycle were treated with non-specific TFOs, or the specific TFO without photoactivation, or mock electroporated and UVA treated. No increase in mutation frequency relative to cells that received no treatment was observed in any of these experiments. The efficiency of electroporation was measured in G₀/G₁ and S phase cells using a GFP reporter plasmid assay. A modest difference in electroporation efficiency was observed in S phase (60-70% positive cells) as compared to G₀/G₁ cells (40-50%). The assay was repeated with fluorescent oligonucleotides. Approximately 50% of the G₀/G₁ cells showed nuclear fluorescence vs 80% for those in the S phase, again too small to explain the differences in FIG. 9.

The Aprt gene has polypurine:polypyrimidine elements, similar, but not identical, to the Exon 5 target in Hprt. There are straightforward selection protocols for identifying cells with mutations in Aprt. This assay was used to monitor the specificity of the TFOs designed for the Hprt target. Experiments indicated that while AE-07 treatment mutagenized Hprt it had no effect on Aprt (14). This experiment with cells in mid S phase yielded no increase over background in the frequency of cells with mutations in the Aprt gene (not shown).

The results of these experiments suggested that the differences in pso-TFO activity in quiescent and S phase cells were not artifacts of electroporation efficiency differences or due to a loss of target specificity (with an attendant increase in random mutagenesis) in S phase. The indifference to cell cycle position of mutagenesis by free psoralen indicated that there was a fundamental difference between the two reagents. Two nonexclusive explanations for the variability of the TFO targeted mutagenesis appeared possible. The first assumed that the frequency of TFO targeted crosslinks was the same in G₀/G₁ and S phase and that the kinds of mutations, or perhaps the efficiency of mutagenesis of the TFO crosslinks varied. Alternatively, the frequency of TFO mediated crosslink formation might be different in G₀/G₁ and S phase.

Kinds and frequency of mutations induced by pso-TFO: The published spectra of psoralen-induced mutations in Hprt are dominated by base substitutions (113, 114). Experiments with mutation reporter plasmids carrying TFO-psoralen crosslinks in the Hprt target sequence embedded in the supF reporter gene indicate that the mutation profiles also included many examples of base substitutions. Almost all were located at T of the 5′ TA crosslink site (data not shown, but see (43) for schematic of the reporter gene). Targeted point mutations were also the major event in earlier studies with psoralen linked TFOs and supF reporter genes (15, 118). However, analysis of pso-TFO targeted mutations at the endogenous chromosomal Hprt target site revealed that ˜90% of thioguanine resistant clones carried deletions in the target region extending into Exon 5 (16, 43), and no point mutations were observed at the T of the 5′ TA crosslink site. This disparity could result from marked differences in the processing of the pso-TFO crosslink in the chromosomal target as compared to the shuttle vector plasmid (and the free psoralen), or might simply reflect the failure of the thioguanine resistance assay to report base substitutions at this site.

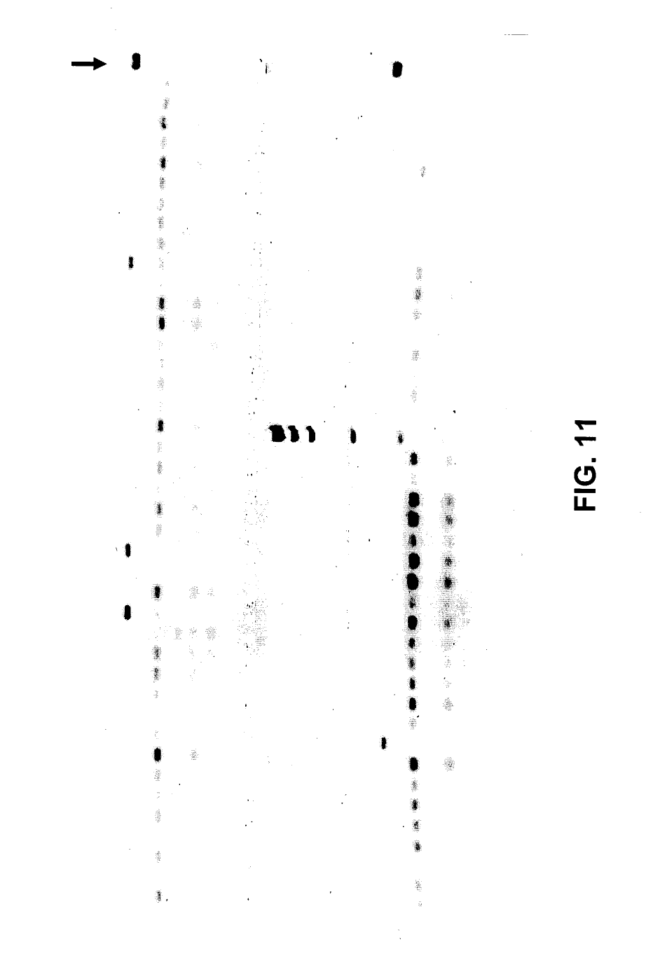

The latter possibility was investigated by isolating DNA from colonies chosen at random following pso-TFO/UVA treatment (no selection was applied). The PCR products of the 14E5 target region from these clones were digested with XbaI whose recognition site is coincident with the crosslink target site (FIG. 1a). 800 colonies from S phase experiments were screened, yielding 54 digestion-resistant clones (6.8%)(FIG. 11). The analysis was performed on G₀/G₁ colonies and 6 of 731 were resistant (0.8%). Sequence analysis of resistant PCR fragments (43 S phase and 4 G₀/G₁) indicated that 41/43 were T->C at the T of the 5′ TA crosslink site (the others were point mutations at adjacent bases within the XbaI site). Two hundred colonies from mock transfected/UVA treated cells were also analyzed. There were no XbaI resistant clones. These results showed that the actual frequency of targeted mutations was much higher (30 fold) than reported by the thioguanine selection. However these results confirmed the disparity between the G₀/G₁ and S phase mutation frequencies.

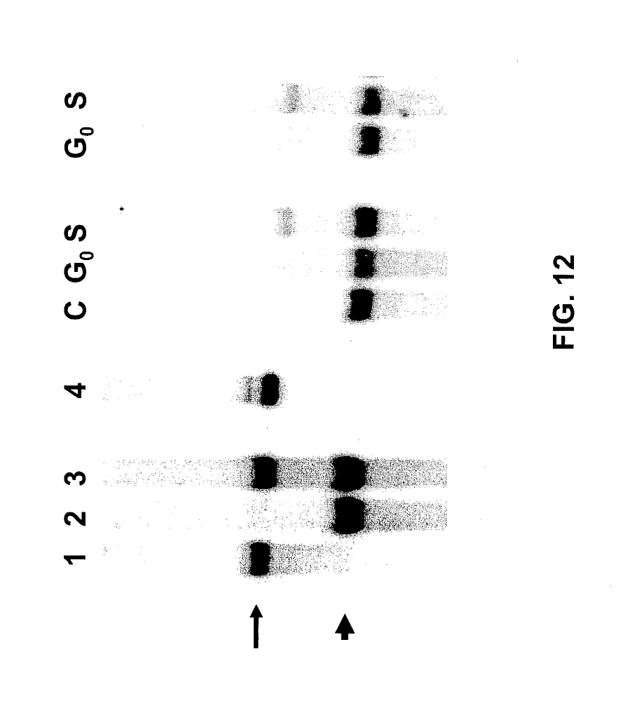

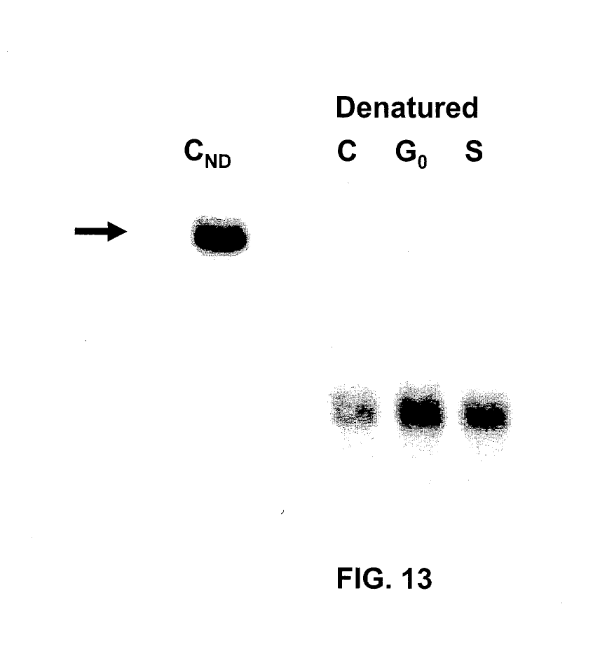

Pso-TFO mediated crosslinking in synchronized cells: The difference in targeted mutation frequency between quiescent and S phase cells could be due to cell cycle variability in mutagenesis functions (119) or a difference in the efficiency of pso-TFO crosslink formation. Thus, G₀/G₁ and S phase cells were treated with the pso-TFO and UVA, and then isolated total genomic DNA. The classical denaturation resistance technique was used to measure the level of crosslinks in a restriction fragment containing the target sequence (120). The DNA was digested with EcoRI, and the digests denatured and electrophoresed on neutral agarose gels. The gels were blotted onto a nylon filter and hybridized with a probe specific for the 2 kb fragment containing the target sequence. In initial control experiments purified genomic DNA was incubated with the pso-TFO and UVA treated in vitro, restricted, and then either run separately or mixed with an equal amount of untreated DNA. The hybridization pattern showed clear resolution between the crosslinked and non-crosslinked samples, run separately or in mixture (FIG. 12). The hybridization signal of the crosslinked fragment was about 50% of the non-crosslinked control, reflecting the reduced efficiency of hybridization, and/or the reduced retention on the filter, of crosslinked DNA. The analysis of the DNA from the treated cells showed a greater extent of crosslinking in S phase than in G₀/G₁. Quantitation by phosphorimager indicated that about 19±2% of the S phase DNA was crosslinked (7 experiments), while the Go level was 4±2%. To control for nonspecific crosslinking, the blots were stripped and rehybridized with a probe to a 3 kb fragment of the Dhfr gene (FIG. 13). There were no denaturation resistant fragments in any of the samples, suggesting that the resistance was specific to the fragment containing the target sequence.

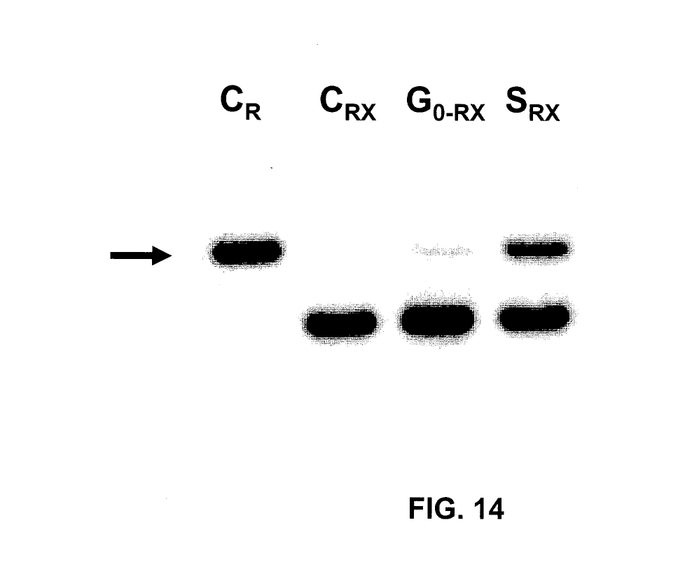

The difference in targeted TFO-psoralen adducts in cells in S phase or G₀/G₁ was also shown by restriction digestion protection. DNA from treated cells was digested with EcoRI and XbaI, followed by blotting and hybridization. The level of the XbaI resistant fragment was several fold higher in the S phase (25-30% protection) sample than Go/GI (4-5%) (FIG. 14). The greater signal from this assay may reflect the inhibition of the restriction enzyme by both psoralen mono adducts and crosslinks, while only crosslinks are reported by the denaturation resistance assay.

Throughout this application, various publications are referenced. The disclosures of these publications in their entireties are hereby incorporated by reference into this application in order to more fully describe the state of the art to which this description pertains.

It will be apparent to those skilled in the art that various modifications and variations can be made in the present compounds, compositions and methods without departing from the scope or spirit of the disclosure. Other embodiments of the compounds, compositions and methods will be apparent to those skilled in the art from consideration of the specification and practice of the procedures disclosed herein. It is intended that the specification and examples be considered as exemplary only, with a true scope and spirit of the invention being indicated by the following claims.

### IV. References

The following references can be referred to in the specification and each one is specifically herein incorporated by reference.

1. Felsenfeld, G., Davies, D. R., and Rich, A. (1957) J. Am. Chem. Soc. 79, 2023-2024.

2. Moser, H. E. and Dervan, P. B. (1987) Science 238, 645-650.

3. Thuong, N. T. and Helene, C. (1993) Angewandte Chemie, Intl. Ed., Eng 32, 666-690.

4. Manor, H., Rao, B. S., and Martin, R. G. (1988) J. Mol. Evol. 27, 96-101.

5. Schroth, G. P. and Ho, P. S. (1995) Nucleic Acids Res. 23, 1977-1983.

6. Behe, M. J. (1995) Nucleic Acids Res. 23, 689-695.

7. Radhakrishnan, I. and Patel, D. J. (1994) Biochemistry 33, 11405-11416.

8. Frank-Kamenetskii, M. D. and Mirkin, S. M. (1995) Annu. Rev. Biochem. 64, 65-95.

9. Fossella, J. A., Kim, Y. J., Shih, H., Richards, E. G., and Fresco, J. R. (1993) Nucleic Acids Res. 21, 4511-4515.

10. Letai, A. G., Palladino, M. A., Fromm, E., Rizzo, V., and Fresco, J. R. (1988) Biochemistry 27, 9108-9112.

11. Helene, C., Thuong, N. T., and Harel-Bellan, A. (1992) Ann. N. Y Acad. Sci. 660, 27-36.

12. Vasquez, K. M. and Wilson, J. H. (1998) Trends Biochem. Sci. 23, 4-9.

13. Maher, L. J., III (1996) Cancer Invest 14, 66-82.

14. Neidle, S. (1997) Anticancer Drug Des 12, 433-442.

15. Wang, G., Levy, D. D., Seidman, M. M., and Glazer, P. M. (1995) Mol. Cell Biol. 15, 1759-1768.

16. Majumdar, A., Khorlin, A., Dyatkina, N., Lin, F. L., Powell, J., Liu, J., Fei, Z., Khripine, Y., Watanabe, K. A., George, J., Glazer, P. M., and Seidman, M. M. (1998) Nat. Genet. 20, 212-214.

17. Vasquez, K. M., Narayanan, L., and Glazer, P. M. (2000) Science 290, 530-533.

18. Asensio, J. L., Lane, A. N., Dhesi, J., Bergqvist, S., and Brown, T. (1998) J. Mol. Biol. 275, 811-822.

19. Blume, S. W., Lebowitz, J., Zacharias, W., Guarcello, V., Canfield, C. A., Ebbinghaus, S. W., Bates, P., Jones Jr, D. E., Trent, J., Vigneswaran, N., and Miller, D. M. (1999) Nucleic Acids Res. 27, 695-702.

20. Pesco, J., Salmon, J. M., Vigo, J., and Viallet, P. (2001) Anal. Biochem. 290, 221-231.

21. Lee, J. S., Woodsworth, M. L., Latimer, L. J., and Morgan, A. R. (1984) Nucleic Acids Res. 12, 6603-6614.

22. Povsic, T. J. a. P.B.D. (1989) J. Am. Chem. Soc. 111, 3059-3061.

23. Miller, P. S., Bi, G., Kipp, S. A., Fok, V., and DeLong, R. K. (1996) Nucleic Acids Res. 24, 730-736.

24. Lacroix, L., Lacoste, J., Reddoch, J. F., Mergny, J. L., Levy, D. D., Seidman, M. M., Matteucci, M. D., and Glazer, P. M. (1999) Biochemistry 38, 1893-1901.

25. Lacroix, L., Arimondo, P. B., Takasugi, M., Helene, C., and Mergny, J. L. (2000) Biochem. Biophys. Res. Commun. 270, 363-369.

26. Roberts, R. W. and Crothers, D. M. (1992) Science 258, 1463-1466.

27. Escude, C., Sun, J. S., Rougee, M., Garestier, T., and Helene, C. (1992) C. R. Acad. Sci. III 315, 521-525.

28. Shimizu, M., Konishi, A., Shimada, Y., Inoue, H., and Ohtsuka, E. (1992) FEBS Lett. 302, 155-158.

29. Torigoe, H., Hari, Y., Sekiguchi, M., Obika, S., and Imanishi, T. (2001) J. Biol. Chem 276, 2354-2360.

30. Kukreti, S., Sun, J. S., Garestier, T., and Helene, C. (1997) Nucleic Acids Res. 25, 4264-4270.

31. Arya, D. P. and Bruice, T. C. (1999) Proc. Natl. Acad. Sci. U.S.A. 96, 4384-4389.

32. Escude, C., Giovannangeli, C., Sun, J. S., Lloyd, D. H., Chen, J. K., Gryaznov, S. M., Garestier, T., and Helene, C. (1996) Proc. Natl. Acad. Sci. U.S. A 93, 4365-4369.

33. Dagle, J. M. and Weeks, D. L. (1996) Nucleic Acids Res. 24, 2143-2149.

34. Ehrenmann, F., Vasseur, J. J., and Debart, F. (2001) Nucleosides Nucleotides Nucleic Acids 20, 797-799.

35. Vasquez, K. M., Dagle, J. M., Weeks, D. L., and Glazer, P. M. (2001) J. Biol. Chem. 276, 38536-38541.

36. Bijapur, J., Keppler, M. D., Bergqvist, S., Brown, T., and Fox, K. R. (1999) Nucleic Acids Res. 27, 1802-1809.

37. Rajeev, K. G., Jadhav, V. R., and Ganesh, K. N. (1997) Nucleic Acids Res. 25, 4187-4193.

38. Sund, C., Puri, N., and Chattopadhyaya, J. (1996) Tetrahedron 52, 12275-12290.

39. Cuenoud, B., Casset, F., Husken, D., Natt, F., Wolf, R. M., Altmann, K. H., Martin, P., and Moser, H. E. (1998) Angewandte Chemie, Intl. Ed., Eng 37, 1288-1291.

40. Blommers, M. J., Natt, F., Jahnke, W., and Cuenoud, B. (1998) Biochemistry 37, 17714-17725.

41. Stutz, A. M., Hoeck, J., Natt, F., Cuenoud, B., and Woisetschlager, M. (2001) J. Biol. Chem 276, 11759-11765.

42. Carlomagno, T., Blommers, M. J., Meiler, J., Cuenoud, B., and Griesinger, C. (2001) J. Am. Chem Soc. 123, 7364-7370.

43. Puri, N., Majumdar, A., Cuenoud, B., Natt, F., Martin, P., Boyd, A., Miller, P.S., and Seidman, M. M. (2001) J. Biol. Chem 276, 28991-28998.

44. Pon, R. T., Yu, S., and Sanghvi, Y. S. (1999) Bioconjug. Chem 10, 1051-1057.

45. Manoharan, M., Prakash, T. P., Barber-Peoc'h, I., Bhat, B., Vasquez, G., Ross, B. S., and Cook, P. D. (2002) J. Org. Chem 64, 6468-6472.

46. Lin, F. L., Majumdar, A., Klotz, L. C., Reszka, A. P., Neidle, S., and Seidman, M. M. (2000) J. Biol. Chem. 275, 39117-39124.

47. Peden, K. W., Pipas, J. M., Pearson-White, S., and Nathans, D. (1980) Science 209, 1392-1396.

48. Seidman, M. M., Dixon, K., Razzaque, A., Zagursky, R. J., and Berman, M. L. (1985) Gene 38, 233-237.

49. Orren, D. K., Petersen, L. N., and Bohr, V. A. (1995) Mol. Cell Biol. 15, 3722-3730.

50. Asensio, J. L., Carr, R., Brown, T., and Lane, A. N. (1999) J. Am. Chem. Soc. 121, 11063-11070.

51. Kamiya, M., Torigoe, H., Shindo, H., and Sarai, A. (1996) J. Am. Chem Soc. 118, 4532-4538.

52. Obika, S., Uneda, T., Sugimoto, T., Nanbu, D., Minami, T., Doi, T., and Imanishi, T. (2001) Bioorg. Med. Chem. 9, 1001-1011.

53. Leumann, C. J. (2002) Bioorg. Med. Chem. 10, 841-854.

54. Mills, M., Arimondo, P. B., Lacroix, L., Garestier, T., Klump, H., and Mergny, J. L. (2002) Biochemistry 41, 357-366.

55. Svinarchuk, F., Paoletti, J., and Malvy, C. (1995) J. Biol. Chem. 270, 14068-14071.

56. Alunni-Fabbroni, M., Pirulli, D., Manzini, G., and Xodo, L. E. (1996) Biochemistry 35, 16361-16369.

57. Sugimoto, N., Wu, P., Hara, H., and Kawamoto, Y. (2001) Biochemistry 40, 9396-9405.

58. Rougee, M., Faucon, B., Mergny, J. L., Barcelo, F., Giovannangeli, C., Garestier, T., and Helene, C. (1992) Biochemistry 31, 9269-9278.

59. Shindo, H., Torigoe, H., and Sarai, A. (1993) Biochemistry 32, 8963-8969.

60. Xodo, L. E. (1995) Eur. J. Biochem. 228, 918-926.

61. Porschke, D. and Eigen, M. (1971) J. Mol. Biol. 62, 361-381.

62. Prevot-Halter, I. and Leumann, C. J. (1999) Bioorg. Med. Chem. Lett. 9, 2657-2660.

63. Gowers, D. M. and Fox, K. R. (1999) Nucleic Acids Res. 27, 1569-1577.

64. Verma, S. and Miller, P. S. (1996) Bioconjug. Chem. 7, 600-605.

65. Chen D. L., McLaughlin L. W. Use of pK(a) differences to enhance the formation of base triplets involving C-G and G-C base pairs. J Org. Chem. Nov. 3, 2000;65(22):7468-74.

66. Cassidy S. A., Slickers P., Trent J. O., Capaldi D. C., Roselt P. D., Reese C. B., Neidle S., Fox K. R. Recognition of GC base pairs by triplex forming oligonucleotides containing nucleosides derived from 2-aminopyridine. Nucleic Acids Res. Dec. 15, 1997;25(24):4891-8

67. Bates P. J., Laughton C. A., Jenkins T. C, Capaldi D. C., Roselt P. D., Reese C. B., Neidle S. Efficient triple helix formation by oligodeoxyribonucleotides containing alpha- or beta-2-amino-5-(2-deoxy-D-ribofuranosyl) pyridine residues. Nucleic Acids Res. Nov. 1, 1996;24(21):4176-84.

68. Chin T M, Lin S B, Lee S Y, Chang M L, Cheng A Y, Chang F C, Pastemack L, Huang D H, Kan L S. “Paper-clip” type triple helix formation by 5′-d-(TC)3Ta(CT)3Cb(AG)3 (a and b=0-4) as a function of loop size with and without the pseudoisocytosine base in the Hoogsteen strand. Biochemistry. Oct. 10, 2000;39(40):12457-64.

69. Chu C. K., Wempen I., Watanabe K. A., Fox J. J. Nucleosides. 100. General synthesis of pyrimidine C-5 nucleosides related to pseudouridine. Synthesis of 5-(beta-D-ribofuranosyl) isocytosine (pseudoisocytidine),5-(beta-D-ribofuranosyl) 2-thiouracil(2-thiopseudouridine) and 5-(beta-D ribofuranosyl)uracil(pseudouridine). J. Org. Chem. Aug. 20, 1976;41(17):2793-7.

70. Burchenal J. H., Ciovacco K., Kalaher K., O'Toole T., Kiefner R., Dowling M. D., Chu C. K., Watanabe K. A., Wempen I., Fox J. J. Antileukemic effects of pseudoisocytidine, a new synthetic pyrimidine C-nucleoside. Cancer Res. 1976 April; 36(4): 1520-3.

71. Verma S., Miller P.S. Interactions of cytosine derivatives with T.A interruptions in pyrimidine.purine.pyrimidine DNA triplexes. Bioconjug. Chem. 1996 Sep-Oct; 7(5):600-5.

72. Huang C. Y., Bi G., Miller P. S. Triplex formation by oligonucleotides containing novel deoxycytidine derivatives. Nucleic Acids Res. Jul. 1, 1996;24(13):2606-13.

73. Miller P. S., Bi G., Kipp S. A., Fok V., DeLong R. K. Triplex formation by a psoralen-conjugated oligodeoxyribonucleotide containing the base analog 8-oxo-adenine. Nucleic Acids Res. Feb. 15, 1996;24(4):730-6.

74. Miller P. S., Bhan P., Cushman C. D., Trapane T. L. Recognition of a guanine-cytosine base pair by 8-oxoadenine. Biochemistry Jul. 28, 1992;31(29):6788-93.

75. Miller P. S., Cushman C. D. Selective modification of cytosines in oligodeoxyribonucleotides. Bioconjug Chem. 1992 Jan-Feb; 3(1):74-9.

76. Nair V., Sosnouski D. S., Zhu Q. Isonucleosides incorporating universal bases. Nucleosides Nucleotides Nucleic Acids. 2001 Apr-Jul; 20(4-7):735-8.

77. Oliver J. S., Parker K. A., Suggs J. W. Effect of the universal base 3-nitropyrrole on the selectivity of neighboring natural bases. Org. Lett. Jun. 28, 2001;3(13):1977-80.

78. Cubero E., Guimil-Garcia R., Luque F. J., Eritja R., Orozco M. The effect of amino groups on the stability of DNA duplexes and triplexes based on purines derived from inosine. Nucleic Acids Res. Jun. 15, 2001;29(12):2522-34.

79. Loakes D. Survey and summary: The applications of universal DNA base analogues. Nucleic Acids Res. Jun. 15, 2001;29(12):2437-47. Review.

80. Pirrung M. C., Zhao X., Harris S. V. A universal, photocleavable DNA base: nitropiperonyl 2′-deoxyriboside. J. Org. Chem. Mar. 23, 2001;66(6):2067-71.

81. Seela F., Debelak H. The N(8)-(2′-deoxyribofuranoside) of 8-aza-7-deazaadenine: a universal nucleoside forming specific hydrogen bonds with the four canonical DNA constituents. Nucleic Acids Res. 2000 Sep 1;28(17):3224-32.

82. Kunitsyn A., Kochetkova S., Kolganova N., Tishchenko E., Gottikh B., Florentiev V. Stabilizing effect of 5-nitroindole (universal base) on DNA duplexes immobilized on gel matrix. J. Biomol. Struct. Dyn. 1997 Dec; 15(3):597-603.

83. Amosova O., George J., Fresco J. R. Effect of the 1-(2′-deoxy-beta-D-ribofuranosyl)-3-nitropyrrole residue on the stability of DNA duplexes and triplexes. Nucleic Acids Res. May 15, 1997;25(10):1930-4.

84. Rahman M. S., Humayun M. Z. Nebularine (9-2′-deoxy-beta-D-ribofuranosylpurine) has the template characteristics of adenine in vivo and in vitro. Mutat. Res. Jul. 3, 1997;377(2):263-8.

85. Loakes D., Brown D. M. 5-Nitroindole as an universal base analogue. Nucleic Acids Res. 1994 Oct 11;22(20):4039-43.

86. “Nucleoside Mimetics” C. Simons, Gordon and Breach Science Publishers, Amsetrdam, Netherlands, 2001.

87. Duzgunes N., Simoes S., Slepushkin V., Pretzer E., Rossi J. J., De Clercq E., Antao V. P., Collins M. L., de Lima M. C. Enhanced inhibition of HIV-1 replication in macrophages by antisense oligonucleotides, ribozymes and acyclic nucleoside phosphonate analogs delivered in pH-sensitive liposomes. Nucleosides Nucleotides Nucleic Acids. (2001) 20(4-7): 515-23.

88. Chaput J. C., Switzer C. Nonenzymatic oligomerization on templates containing phosphodiester-linked acyclic glycerol nucleic acid analogues. J. Mol. Evol. 2000 Nov; 51(5):464-70.

89. Ramasamy K. S., Stoisavljevic V. Synthesis and biophysical studies of modified oligonucleotides containing acyclic amino alcohol nucleoside analogs. Nucleosides Nucleotides. (1999) 18(8):1845-61.

90. Van Aerschot A., Rozenski J., Loakes D., Pillet N., Schepers G., Herdewijn P. An acyclic 5-nitroindazole nucleoside analogue as ambiguous nucleoside. Nucleic Acids Res. (1995) 23(21):4363-70.

91. Merle Y., Bonneil E., Merle L., Sagi J., Szemzo A. Acyclic oligonucleotide analogues. Int. J Biol. Macromol. (1995) Oct; 17(5):239-46.

92. Nielsen P., Dreioe L. H., Wengel J. Synthesis and evaluation of oligodeoxynucleotides containing acyclic nucleosides: introduction of three novel analogues and a summary. Bioorg. Med. Chem. (1995) Jan; 3(1):19-28.

93. Merle L., Spach G., Merle Y., Sagi J., Szemzo A. Some biochemical properties of an acyclic oligonucleotide analogue. A plausible ancestor of the DNA? Orig. Life Evol. Biosph. (1993) (2):91-103.

94. Visscher J, Schwartz A W. Oligomerization of cytosine-containing nucleotide analogs in aqueous solution. J. Mol. Evol. (1990) 30(1):3-6.

95. Visscher J, Schwartz A W. Template-directed synthesis of acyclic oligonucleotide analogues. J. Mol. Evol. 1988 Dec-1989 February; 28(1-2):3-6.

96. Sawai, M., Takase, K., Teraoka, H., and Tsukada, K. (1990) Exp. Cell Res. 187, 4-10.

97. Kopel, V., Pozner, A., Baran, N., and Manor, H. (1996) Nucleic Acids Res. 24, 330-335.

98. Brosh, R. M., Jr., Majumdar, A., Desai, S., Hickson, I. D., Bohr, V. A., and Seidman, M. M. (2000) J. Biol. Chem. 276, 3024.

99. Saha, A., Wittmeyer, J., and Cairns, B. R. (2002) Genes Dev. 16, 2120-2134.

100. Steen, A. M., Sahlen, S., and Lambert, B. (1991) Biochim. Biophys. Acta 1088, 77-85.

101. Westin, L., Blomquist, P., Milligan, J. F., and Wrange, O. (1995) Nucleic Acids Res. 23, 2184-2191.

102. Lorch, Y., Zhang, M., and Kornberg, R. D. (2001) Mol. Cell 7, 89-95.

103. Gavin, I., Horn, P. J., and Peterson, C. L. (2001) Mol. Cell 7, 97-104.

104. Peterson C. L. (2002) EMBO Rep. 3, 319-322.

105. Jenuwein, T. and Allis, C. D. (2001) Science 293, 1074-1080.

106. Jiralerspong, S. and Patel, P. I. (1996) Proc. Soc. Exp. Biol. Med. 212, 116-127.

107. Dyson, N. (1998) Genes Dev. 12, 2245-2262.

108. Espinas, M. L., Jimenez-Garcia, E., Martinez-Balbas, A., and Azorin, F. (1996) J. Biol. Chem. 271, 31807-31812.

109. Brown, P. M. and Fox, K. R. (1999) Eur. J. Biochem. 261, 301-310.

110. Brown, P. M., Madden, C. A., and Fox, K. R. (1998) Biochemistry 37, 16139-16151.

111. Hearst, J. E. (1989) Chem Res. Toxicol. 2, 69-75.

112. Puri, N., Majumdar, A., Cuenoud, B., Natt, F., Martin, P., Boyd, A., Miller, P. S., Seidman, M. M. (2002) Biochemistry 41, 7716-7724.

113. Laquerbe, A., Guillouf, C., Moustacchi, E., and Papadopoulo, D. (1995) J. Mol. Biol. 254, 38-49.

114. Chiou, C. C. and Yang, J. L. (1995) Carcinogenesis 16, 1357-1362.

115. Rayman, J. B., Takahashi, Y., Indjeian, V. B., Dannenberg, J. H., Catchpole, S., Watson, R. J., te Riele H., and Dynlacht, B. D. (2002) Genes Dev. 16, 933-947.

116. Ogawa, H., Ishiguro, K., Gaubatz, S., Livingston, D. M., and Nakatani, Y. (2002) Science 296, 1132-1136.

117. Grigoriev, M., Praseuth, D., Guieysse, A. L., Robin, P., Thuong, N. T., Helene, C., and Harel-Bellan, A. (1993) Proc. Natl. Acad. Sci. U.S.A. 90, 3501-3505.

118. Raha, M., Lacroix, L., and Glazer, P. M. (1998) Photochem. Photobiol. 67, 289-294.

119. Hendrickson, E. A. (1997) Am. J. Hum. Genet. 61, 795-800.

120. Hartley, J. A., Souhami, R. L., and Berardini, M. D. (1993) J. Chromatogr. 618, 277-288.

121. Cariello, N. F., Craft, T. R., Vrieling, H., van Zeeland, A. A., Adams, T., and Skopek, T. R. (1992) Environ. Mol. Mutagen. 20, 81-83.

We claim:

1. A method for modifying a nucleotide sequence in the genome of a cell, comprising:
providing a cell in S-phase of the cell cycle; and
contacting the cell with a DNA modifying molecule that modifies the nucleotide sequence.

2. The method of claim 1, wherein contacting the cell comprises activating the DNA modifying molecule to modify the nucleotide sequence.

3. The method of claim 2, wherein the DNA modifying molecule is activatable by radiation, and activating the DNA modifying molecule comprises irradiating the molecule with radiation.

4. The method of claim 3, wherein the radiation is ultraviolet radiation.

5. The method of claim 1, wherein the cell is contacted with the DNA modifying molecule at a cell cycle phase wherein DNA modification by the DNA modifying molecule occurs at a higher frequency relative to a second cell cycle phase.

6. The method of claim 1, wherein the cell is contacted with the DNA modifying molecule in mid to late S phase.

7. The method of claim 1, wherein providing a cell comprises providing a population of cells.

8. The method of claim 7, further comprising synchronizing the population of cells to yield a synchronized cell population, and contacting the cell comprises contacting the synchronized cell population with the DNA modifying molecule.

9. The method according to claim 8, wherein at least 50% of the synchronized cell population is synchronized in S phase when the synchronized cell population is contacted with the DNA modifying molecule.

10. The method according to claim 9, wherein at least 75% of the synchronized cell population is synchronized in S phase when the synchronized cell population is contacted with the DNA modifying molecule.

11. The method of claim 1, wherein the cell is a human cell.

12. The method of claim 1, wherein the cell is a fertilized egg cell from an animal selected from the group consisting of a mouse, hamster, sheep, pig, rabbit, or cow.

13. The method of claim 1, wherein the cell is a mouse cell selected from the group consisting of a blastomere cell, an eight-cell embryo cell, a blastocoele cell, a midgestation cell embryo cell, or an embryonic stem cell.

14. The method of claim 1, wherein the DNA modifying molecule comprises a DNA targeting agent selected from the group consisting of peptide nucleic acids, polyamides, triplex forming oligonucleotides, zinc finger proteins and combinations thereof.

15. The method of claim 1, wherein the DNA modifying molecule comprises a modified oligonucleotide.

16. The method of claim 15, wherein the modified oligonucleotide is a triplex forming oligonucleotide.

17. The method of claim 15, wherein the modified oligonucleotide comprises at least one 2′-O-alkylated residue.

18. The method of claim 17, wherein the 2′-O-alkylated residue is a 2′-aminoalkoxy residue.

19. The method of claim 17, wherein the at least one 2′-O-alkylated residue is a pyrimidine residue.

20. The method of claim 15, wherein the modified oligonucleotide comprises from 10 to 25 residues.

21. The method of claim 20, wherein the modified oligonucleotide comprises no more than four 2′-O-alkylated residues.

22. The method of claim 21, wherein the modified oligonucleotide comprises multiple contiguous 2′-aminoalkoxy residues.

23. The method of claim 21, wherein the modified oligonucleotide comprises four contiguous 2′-aminoalkoxy residues.

24. The method of claim 15, wherein the modified oligonucleotide comprises at least one unit according to formula I 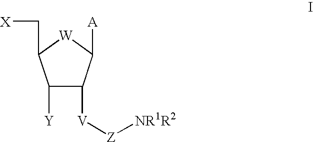
wherein A is a residue of a nucleic acid base;
X and Y are, independently, the same or different residues of an internucleosidic bridging group or a terminal group;
V and W are, independently, oxygen, sulfur, NR³, or CR⁴R⁵;
Z, is an alkyl group, a cycloalkyl group, a heterocyloalkyl group, a hydroxyalkyl group, a halogenated alkyl group, an alkoxyalkyl group, an alkenyl group, an alkynyl group, an aryl group, a heteroaryl group, an aralkyl group, or a combination thereof,
R¹, R², R³, R⁴, and R⁵ are, independently, hydrogen, an alkyl group, a cycloalkyl group, a heterocyloalkyl group, an alkoxy group, a hydroxyalkyl group, a halogenated alkyl group, an alkoxyalkyl group, an alkenyl group, an alkynyl group, an aryl group, a heteroaryl group, an aralkyl group, a hydroxy group, an amine group, an amide, an ester, a carbonate group, a carboxylic acid, an aldehyde, a keto group, an ether group, a halide, a urethane group, a silyl group, or a combination thereof, wherein R¹ and R² can be part of a ring; or
a salt thereof.

25. A cell modified by the method of claim 1.

26. A modified oligonucleotide comprising at least one unit according to formula I 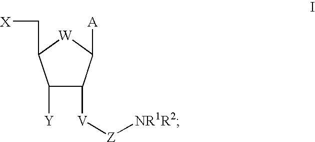
a mutagen covalently attached to the modified oligonucleotide; or
a salt thereof.

27. The oligonucleotide of claim 26, wherein A is a residue of xanthine, hypoxanthine, adenine, 2-aminoadenine, guanine, cytosine, thymine, 6-thioguanine, uracil, 5-methylcytosine, 5-propynyluracil, 5-fluorouracil, 5-propynylcytosine, 2,6-diaminopurine, purine, 7-deazaadenine, 7-deazaguanine, 5-propynyluracil, isoguanine, 2-aminopurine, 6-methyluracil, 4-thiouracil, 2-pyrimidone, N,N-dimethylguanine, bromouracil, aminopyridine, or a functional equivalent thereof.

28. The oligonucleotide of claim 26, wherein A is a pyrimidine residue.

29. The oligonucleotide of claim 26, wherein A is a residue of cytosine or 5-substituted cytosine.

30. The oligonucleotide of claim 26, wherein A is a residue of uridine or 5-substituted uridine.

31. The oligonucleotide of claim 26, wherein X and Y together form a phosphodiester, a phosphorothioate, methylphosphonate, H-phosphonate, or an amide bond between adjacent nucleosides or nucleoside analogs or together form an analog of a phosphodiester bond.

32. The oligonucleotide of claim 26, wherein Z is a lower alkyl group.

33. The oligonucleotide of claim 26, wherein Z is —CH₂CH₂—.

34. The oligonucleotide of claim 26, wherein R¹ and R² are, independently selected from the group consisting of hydrogen, methyl, ethyl, propyl, or phenyl.

35. The oligonucleotide of claim 26, wherein V and W are oxygen.

36. The oligonucleotide of claim 26, wherein A is a residue of 5-methylcytosine or thymine; V and W are oxygen; X and Y together form a phosphodiester bond; Z is —CH₂CH₂—; and R¹ and R² are hydrogen.

37. The oligonucleotide of claim 26, wherein the mutagen comprises a moiety selected from the group consisting of radionuclides, crosslinkers, alkylators, base modifiers, DNA breakers, free radical generators and combinations thereof.

38. The oligonucleotide of claim 26, wherein the mutagen comprises a moiety selected from the group consisting of bleomycin, cyclopropapyrroloindoles, phenanthodihydrodioxins, indolocarbazoles, napthalene diimide, chlorambucil, mitomycin derivatives, enediyne derivatives, hematoporphyrin derivatives, coumarin derivatives, oxazolopyridocarbazole, daunomycine, anthraquinone, acridine orange, cis platinum derivatives, radionuclides, boron agents, and combinations thereof.

39. The oligonucleotide of claim 26, wherein the mutagen comprises an intercalator.

40. The oligonucleotide of claim 26, wherein the mutagen is activatable by irradiation.

41. The oligonucleotide of claim 26, wherein the mutagen comprises a psoralen derivative.

42. The oligonucleotide of claim 26, wherein the oligonucleotide contains from 10 to 25 nucleotides.

43. The oligonucleotide of claim 26, wherein the oligonucleotide comprises multiple units having formula I.

44. The oligonucleotide of claim 43, wherein two or more of the multiple units having formula I are localized to a region of the oligonucleotide that is less than the entire length of the oligonucleotide.

45. The oligonucleotide of claim 43, wherein two or more of the multiple units having formula I are contiguous.

46. The oligonucleotide of claim 44, wherein the region of the oligonucleotide is less than 6 residues in length.

47. The oligonucleotide of claim 23, comprising at least 3 but no more than 4 units having formula I.

48. The oligonucleotide of claim 47, wherein the units having formula I are contiguous.

49. A pharmaceutical composition comprising the oligonucleotide of claim 26 and a pharmaceutically acceptable carrier.

50. A method for mutating a nucleotide sequence in the genome of a cell, comprising contacting the cell with a modified oligonucleotide to produce a mutation of the nucleotide sequence, wherein the modified oligonucleotide comprises at least one unit according to formula I, or a salt thereof.

51. The method of claim 50, wherein mutating the nucleotide sequence comprises introducing a deletion, insertion, substitution, strand break, adduct formation, gene conversion, or recombination of a novel sequence.

52. The method of claim 50, wherein the cell is human.

53. The method of claim 50, wherein the cell is non-human.

54. The method of claim 50, wherein the cell is a fertilized egg cell from an animal selected from the group consisting of a mouse, hamster, sheep, pig, rabbit, or cow.

55. The method of claim 50, wherein the cell is a mouse cell selected from the group consisting of a blastomere cell, an eight-cell embryo cell, a blastocoele cell, a midgestation cell embryo cell, or an embryonic stem cell.

56. The method of claim 50, wherein contacting the cell with the modified oligonucleotide comprises contacting a population of cells.

57. The method of claim 56, wherein contacting the population of cells with the modified oligonucleotide comprises synchronizing the population of cells to yield a synchronized cell population.

58. The method of claim 57, wherein at least 50% of the synchronized cell population is synchronized in S phase when the synchronized cell population is contacted with the modified oligonucleotide.

59. The method of claim 57, wherein at least 75% of the synchronized cell population is synchronized in S phase when the synchronized cell population is contacted with the modified oligonucleotide.

60. The method of claim 50, wherein contacting the cell with the modified oligonucleotide comprises contacting the cell with the modified nucleotide when the cell is in mid to late S phase.

61. A cell produced by the method of claim 50.

62. The cell of claim 61, wherein the cell is incorporated in an animal.

63. The cell of claim 62, wherein the animal is a patient and incorporation of the cell ameliorates a medical condition.

64. A cell comprising the modified oligonucleotide of claim 26.

65. A vector comprising the modified oligonucleotide of claim 26.

66. The vector of claim 65, wherein the vector is a nucleic acid vector.

67. The vector of claim 65, wherein the vector is a viral vector.

68. A modified oligonucleotide, comprising:
a first unit according to formula I;
at least a second unit according to formula I, wherein X or Y comprise a mutagenic group, and V, W, Z, R¹, R², R³, R⁴, and R⁵ are selected as above; or
a salt thereof.

69. The modified oligonucleotide according to claim 68, wherein the mutagenic group of the second unit comprises an intercalator.

70. The modified oligonucleotide according to claim 68, wherein the mutagenic group of the second unit comprises a group according to the formula 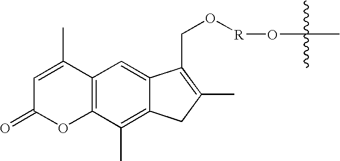
where R is selected from the group consisting of hydrocarbon chains, polyalkylene oxides and polyalkylene imines.

71. A modified oligonucleotide, comprising no more than four units according to formula I, or a salt thereof.

72. The oligonucleotide according to claim 71, wherein the oligonucleotide is from 10 to 25 nucleic acid residues in length.

73. The oligonucleotide according to claim 71, wherein the oligonucleotide includes at least three but no more than four units according to formula I.

74. The oligonucleotide according to claim 71, further comprising a mutagen.

75. The oligonucleotide according to claim 74, wherein the mutagen comprises an intercalator.

76. The oligonucleotide according to claim 74, wherein the mutagen comprises a psoralen derivative.
